(19) United States
(12) Patent Application Publication
(10) Pub. No.: US 2009/0099355 A1
(43) Pub. Date: Apr. 16, 2009

(54) Processes for Production of 4-(Biphenylyl)Azetidin-2-One Phosphonic Acids

(75) Inventors: Stephen Antonelli, Lynn, MA (US); Timothy C. Barden, Salem, MA (US); Peter Lee, Herndon, VA (US); Eduardo J. Martinez, St. Louis, MO (US); Wayne C. Schairer, Westboro, MA (US); John J. Talley, Somerville, MA (US)

Correspondence Address: HESLIN ROTHENBERG FARLEY & MESITI PC, 5 COLUMBIA CIRCLE, ALBANY, NY, 12203, US

(73) Assignee: MICROBIA, INC., Cambridge, MA (US)

(21) Appl. No.: 11/915,241

(22) PCT Filed: May 25, 2006
(86) PCT No.: PCT/US2006/020226

§ 371 Date: Jun. 13, 2008

Related U.S. Application Data
(60) Provisional application No. 60/684,481, filed on May 25, 2005.

(51) Int. Cl. C07D 205/08; C07D 263/22; C07C 211/28; C07F 9/38

(52) U.S. Cl. 540/200; 548/231; 564/336; 562/23

(57) ABSTRACT

The present invention relates to processes for the production of 4-(biphenylyl)azetidin-2-one phosphonic acid derivatives of formula

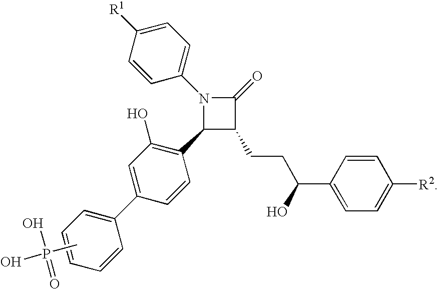

### FIELD OF THE INVENTION

The present invention relates to processes for the production of 4-(biphenylyl)azetidin-2-one phosphonic acid derivatives.

### BACKGROUND OF THE INVENTION

(4′-{(2S,3R)-3-[(3S)-3-(4-Fluorophenyl)-3-hydroxypropyl]-4-oxo-1-phenylazetidin-2-yl}-3′-hydroxybiphenyl-4-yl)phosphonic acid (4-BPA)

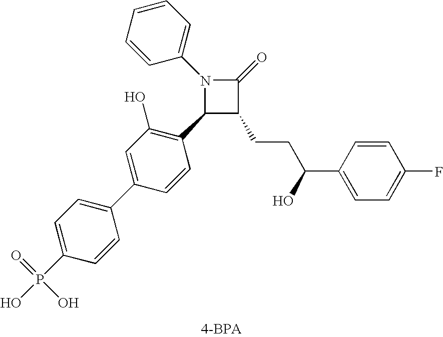

and its isomer, (4′-{(2S,3R)-3-[(3S)-3-(4-fluorophenyl)-3-hydroxypropyl]-4-oxo-1-phenylazetidin-2-yl}-3′-hydroxybiphenyl-3-yl)phosphonic acid (3-BPA)

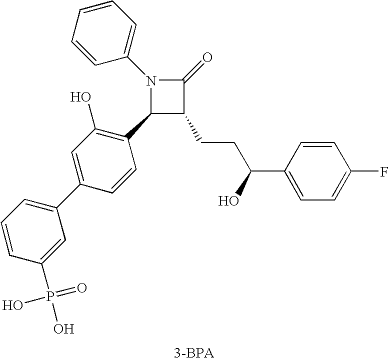

have been shown to be inhibitors of cholesterol absorption. (See copending U.S. application Ser. No. 10/986,570, which is incorporated herein by reference in its entirety. Attention is directed to examples 60, 61 and 127 on pages 90-93 and 119.)

4-BPA and 3-BPA are members of the family of azetidinone cholesterol absorption inhibitors. 1,4-Diphenylazetidin-2-ones and their utility for treating disorders of lipid metabolism are described in U.S. Pat. No. 6,498,156 and PCT application WO02/50027, the disclosures of which are incorporated herein by reference. Perhaps the most well-known member of the class of 1,4-diphenylazetidin-2-one hypocholesterolemics is ezetimibe, which is sold as ZETIA™.

U.S. Pat. Nos. 5,631,365; 6,093,812; 5,306,817 and 6,627,757, for example, disclose and claim processes for the preparation of azetidinone derivatives related to ezetimibe.

The present invention is directed toward a process for preparation of 4-(biphenylyl)azetidin-2-one phosphonic acids.

### SUMMARY OF THE INVENTION

The present invention relates to processes for preparing compounds of the formula I:

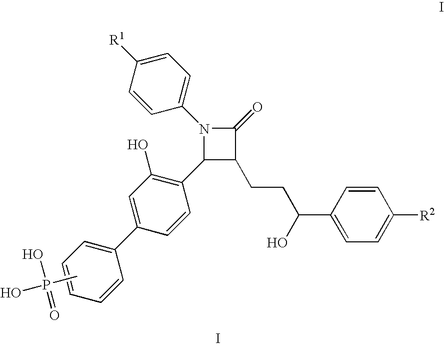

wherein R¹ and R² are chosen independently from H, halogen, —OH, and methoxy.

In a first aspect, the invention relates to a process for preparing Ia

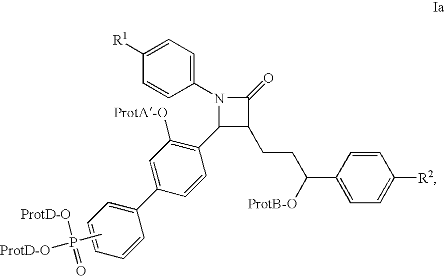

wherein ProtA′-O— is a protecting group for a phenol chosen from an oxymethyl ether, a tertiary alkyl ether, a benzyl ether and a silyl ether; ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and ProtD-O— is HO— or a protecting group for a phosphonic acid chosen from an alkyl ester, a phenyl ester and a benzyl ester. The process comprises reacting a compound of formula IIa

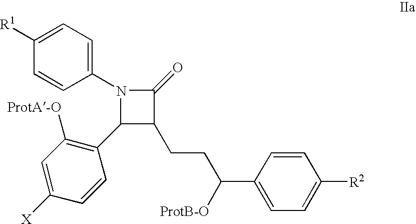

wherein X is chosen from iodine, bromine, chlorine, toluenesulfonyl, methanesulfonyl and trifluoromethanesulfonyl, with a compound of formula III

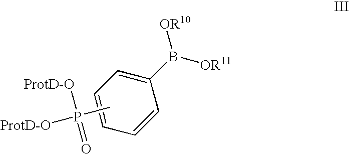

wherein R¹⁰ and R¹¹ are independently selected from H and (C₁-C₆) alkyl, or R¹⁰ and R¹¹ together form a 5-6 membered ring.

Inversely, one may react a compound of formula IIb

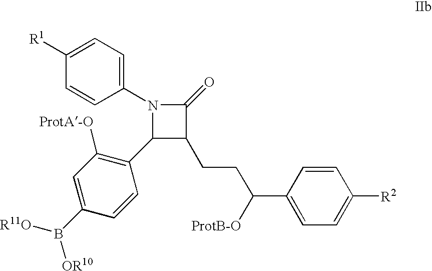

with a compound of formula IIIa

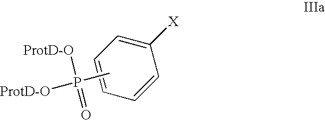

In a second aspect, the invention relates to a process for preparing a compound of structure II

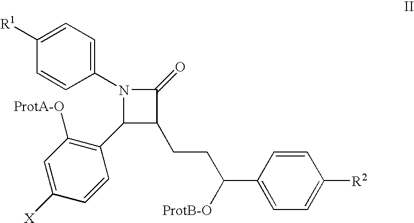

in which ProtA-O— is a protecting group for a phenol chosen from an oxymethyl ether, an allyl ether, a tertiary alkyl ether, a benzyl ether and a silyl ether. The process comprises cyclizing a compound of formula IVa

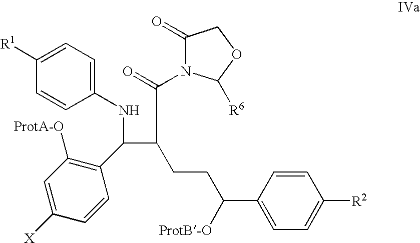

wherein R⁶ is phenyl or benzyl and ProtB′-O— is a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester.

In a third process aspect, the invention relates to a process for preparing a compound of structure IV

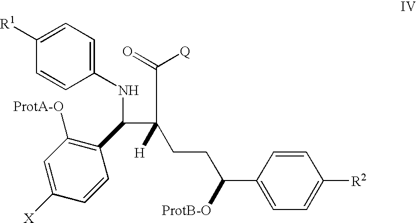

wherein Q is a chiral auxiliary. The chiral auxiliary is chosen from single enantiomers of triphenyl glycol and cyclic and branched nitrogen-containing moieties possessing at least one chiral center. The process comprises reacting a compound of formula V

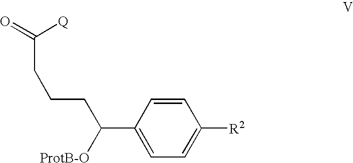

with a compound of formula VI

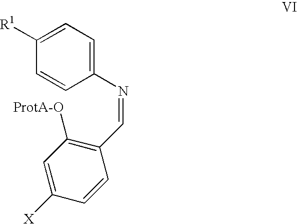

In a fourth process aspect, the invention relates to a process for preparing an imine of formula VI

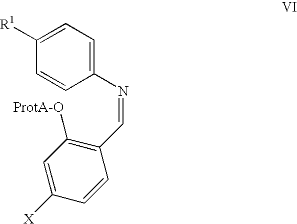

The process comprises (1) reacting a phenol of formula

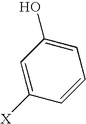

with a source of formaldehyde, followed by (2) Schiff base formation by reacting with an aniline of formula

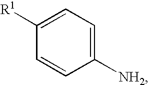

followed by (3) protecting with ProtA.

In combination, the processes of the invention provide an overall process for preparing 4-BPA:

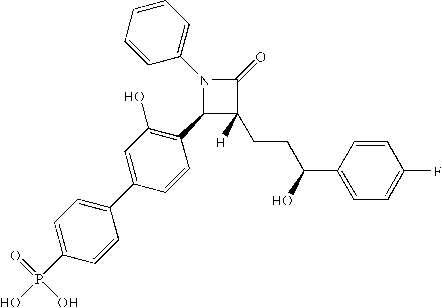

and related biphenyl phosphonic acids.
from

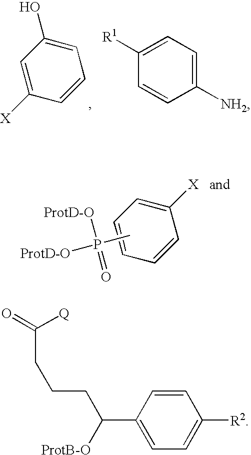

In a product aspect, the invention relates to compounds useful as intermediates in the process.

### DETAILED DESCRIPTION OF THE INVENTION

Throughout this application, various references are cited. The disclosures of each of these publications in their entireties are hereby incorporated by reference as if written herein.

### DEFINITIONS

In this specification the terms and substituents are defined when introduced and retain their definitions throughout.

Alkyl is intended to include linear, branched, or cyclic hydrocarbon structures and combinations thereof. When not otherwise restricted, the term refers to alkyl of 20 or fewer carbons. Lower alkyl refers to alkyl groups of 1, 2, 3, 4, 5 and 6 carbon atoms. Examples of lower alkyl groups include methyl, ethyl, propyl, isopropyl, butyl, s- and t-butyl and the like. Preferred alkyl and alkylene groups are those of C₂₀ or below (e.g. C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀). Cycloalkyl is a subset of alkyl and includes cyclic hydrocarbon groups of 3, 4, 5, 6, 7, and 8 carbon atoms. Examples of cycloalkyl groups include c-propyl, c-butyl, c-pentyl, norbornyl, adamantyl and the like.

C₁ to C₂₀ Hydrocarbon (e.g. C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀) includes alkyl, cycloalkyl, alkenyl, alkynyl, aryl and combinations thereof. Examples include benzyl, phenethyl, cyclohexylmethyl, camphoryl and naphthylethyl. The term “phenylene” refers to ortho, meta or para residues of the formulae:

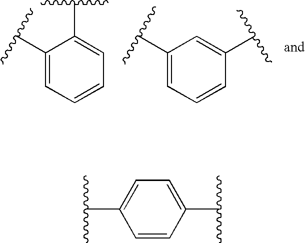

Alkoxy or alkoxyl refers to groups of 1, 2, 3, 4, 5, 6, 7 or 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, ±196, but without the restriction of ¶127(a)], i.e. it refers to compounds in which the oxygen is bonded via a single bond to its adjacent atoms (forming ether bonds). Similarly, thiaalkyl and azaalkyl refer to alkyl residues in which one or more carbons have been replaced by sulfur or nitrogen, respectively. Examples include ethylaminoethyl and methylthiopropyl.

Acyl refers to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include formyl, acetyl, propionyl, isobutyryl, t-butoxycarbonyl, benzoyl, benzyloxycarbonyl and the like. Lower-acyl refers to groups containing one to four carbons.

Aryl and heteroaryl refer to aromatic or heteroaromatic rings, respectively, as substituents. Heteroaryl contains one, two or three heteroatoms selected from O, N, or S. Both refer to monocyclic 5- or 6-membered aromatic or heteroaromatic rings, bicyclic 9- or 10-membered aromatic or heteroaromatic rings and tricyclic 13- or 14-membered aromatic or heteroaromatic rings. Aromatic 6, 7, 8, 9, 10, 11, 12, 13 and 14-membered carbocyclic rings include, e.g., benzene, naphthalene, indane, tetralin, and fluorene and the 5, 6, 7, 8, 9 and 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole.

Arylalkyl means an alkyl residue attached to an aryl ring. Examples are benzyl, phenethyl and the like.

Substituted alkyl, aryl, cycloalkyl, heterocyclyl etc. refer to alkyl, aryl, cycloalkyl, or heterocyclyl wherein up to three H atoms in each residue are replaced with halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The term “halogen” means fluorine, chlorine, bromine or iodine.

Terminology related to “protecting”, “deprotecting” and “protected” functionalities occurs throughout this application. Such terminology is well understood by persons of skill in the art and is used in the context of processes which involve sequential treatment with a series of reagents. In that context, a protecting group refers to a group that is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry [See e.g. Protective Groups in Organic Synthesis by T. W. Greene and P. G. M. Wuts, 2nd Edition; John Wiley & Sons, New York (1991)]. As understood by one skilled in the art, the terms “isopropanol”, “isopropyl alcohol” and “2-propanol” are equivalent and are represented by CAS Registry No: 67-63-0.

The abbreviations Me, Et, Ph, Tf, Ts and Ms represent methyl, ethyl, phenyl, trifluoromethanesulfonyl, toluensulfonyl and methanesulfonyl respectively. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference. As understood by one skilled in the art, the terms “isopropanol”, “isopropyl alcohol” and “2-propanol” are equivalent and represented by CAS Registry No: 67-63-0.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines and single thin lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration.

Thus, the formula XI is intended to encompass both of the pure enantiomers of that pair:

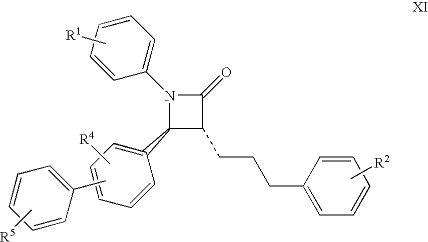

Means either pure 3R,4S:

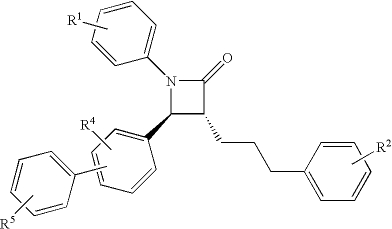

or pure 3S,4R:

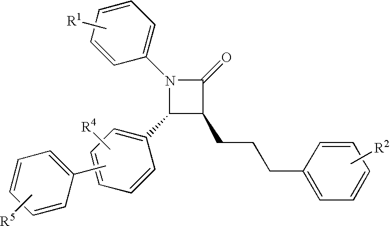

whereas

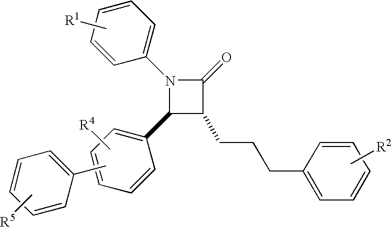

refers to a racemic mixture of R,S and S,R, i.e. having a trans relative configuration on the beta lactam ring.

The term “enantiomeric excess” is well known in the art and is defined for a resolution of ab into a+b as

$$e {e}_{a} = ( \frac{conc . of a - conc . of b}{conc . of a + conc . of b} ) \times 100$$

The term “enantiomeric excess” is related to the older term “optical purity” in that both are measures of the same phenomenon. The value of ee will be a number from 0 to 100, zero being racemic and 100 being pure, single enantiomer. A compound which in the past might have been called 98% optically pure is now more precisely described as 96% ee; in other words, a 90% ee reflects the presence of 95% of one enantiomer and 5% of the other in the material in question.

4-BPA-related compounds of the formula Ia

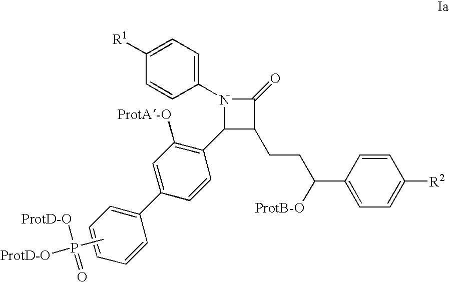

are prepared by reacting a compound of formula IIa

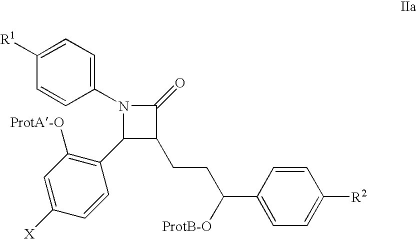

with a compound of formula III

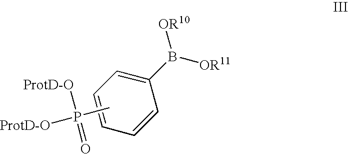

wherein R¹⁰ and R¹¹ are independently selected from H and (C₁-C₆) alkyl, or R¹⁰ and R¹¹ together form a 5-6 membered ring. Alternatively, one may react a compound of formula IIb

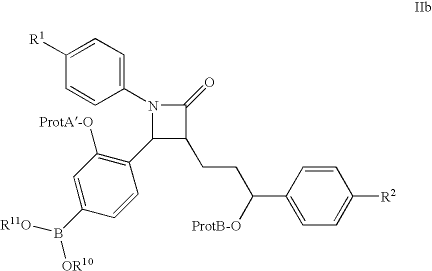

with a compound of formula IIIa

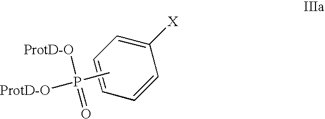

In these processes and compounds, R¹ and R² are chosen from H, halogen, —OH, and methoxy. R¹⁰ and R¹¹ together may form a 5-6 membered ring, for example:

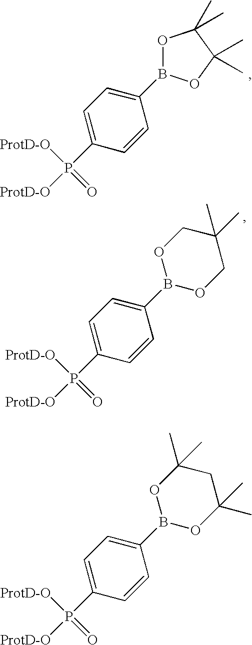

In certain embodiments, R¹ is hydrogen and R² is fluorine and R¹⁰ and R¹¹ together form a dioxaborole. The process for 4-BPA is an example of such an embodiment.

ProtA- is a protecting group for a phenol, and ProtA-O— indicates the protecting group together with the oxygen of the phenol to which it is attached. It is chosen from protecting groups in Greene and Wuts, Chapter 3, that do not require removal with strong acid or base. Examples of such groups include oxymethyl ethers [e.g. MOM and 2-(trimethylsilyl)ethoxymethyl (SEM)], allyl ethers [e.g. allyl ether and 2-methylallyl ether], tertiary alkyl ethers [e.g. t-butyl ether], benzyl ethers [e.g. benzyl ether and various benzyl ether derivatives having substitution on the phenyl ring] and silyl ethers [e.g. trimethylsilyl, t-butyldimethylsilyl, and t-butyldiphenylsilyl].

ProtB- is hydrogen or a protecting group for a benzylic alcohol; ProtB-O— indicates hydrogen or the protecting group together with the oxygen of the benzylic alcohol to which it is attached. For many reactions, including some illustrated below, it is unnecessary to protect the hydroxyl and in these cases, ProtB-O— is HO—. When a protecting group is desired, it is chosen from protecting groups in Greene and Wuts, Chapter 1, pages 17-86, the removal of which does not require strong acid or strong base. Examples include an oxymethyl ether, a tetrahydropyranyl or tetrahydrofuranyl ether, methoxycyclohexyl ether, a methoxybenzyl ether, a silyl ether and an ester [e.g. acetyl or benzoyl].

ProtD- is hydrogen or a protecting group for a phosphonic acid; ProtD-O— indicates hydrogen or the protecting group together with the oxygen of the phosphonic acid to which it is attached. The protecting group may be chosen from any of those well known in the art. Examples include alkyl esters, phenyl esters and benzyl esters.

X is chosen from iodine, bromine, chlorine, toluenesulfonyl, methanesulfonyl and trifluoromethanesulfonyl.

In certain embodiments, ProtA-O— is chosen from methoxymethyl ether, t-butyl ether and benzyl ether; ProtB-O— is chosen from HO—, t-butyldimethylsilyl ether and tetrahydropyranyl ether; and III is

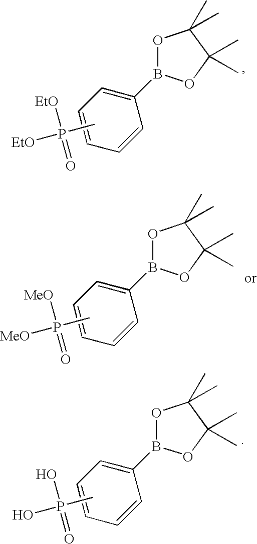

The reaction is brought about in the presence of a phosphine, a palladium salt and a base, for example bis(triphenylphosphine)palladium dichloride and an aqueous solution of an alkali metal hydroxide or carbonate. In one embodiment, R¹ is hydrogen; R² is fluorine; X is bromine; ProtA-O— is benzyl ether; and ProtB-O— is HO—.

After the compound of formula I is synthesized, the protecting groups are cleaved under appropriate conditions to produce the corresponding compounds having a free phenol, free alcohol and/or free phosphonic acid. When the protecting group is, for example, benzyl, hydrogenolysis may be employed for deprotection; when the protecting group is, for example, t-butyldimethylsilyl, tetrabutylammonium fluoride may be employed for deprotection; when the protecting group on phosphorus is, for example, methyl ester, treatment with trialkylsilyl halide may be employed for deprotection.

Thus, for example, one may prepare

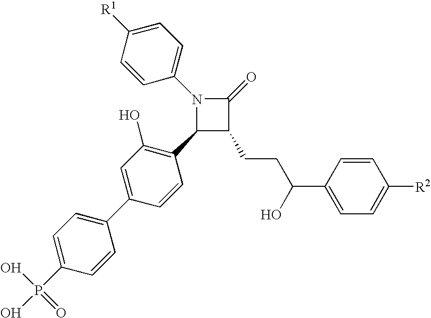

by reacting an azetidinone of formula

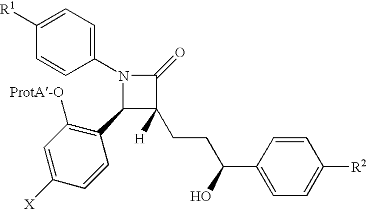

with a dioxaborole of formula

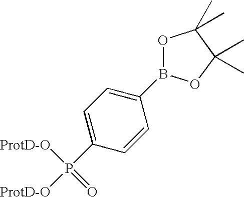

and deprotecting. In this example, ProtD-O— is —OH or methoxy. In a particular embodiment, one may react an azetidinone of formula

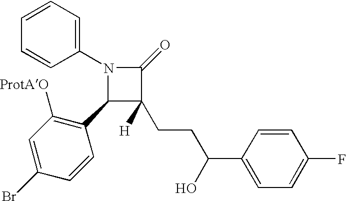

in which ProtA′ is benzyl or TBDMS with a dioxaborole of formula

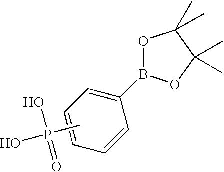

and deprotect. When ProtA′=benzyl the deprotection is accomplished by catalytic hydrogenolysis. When ProtA′=TBDMS the deprotection is accomplished by treatment with fluoride.

The compound of structure II may be synthesized by

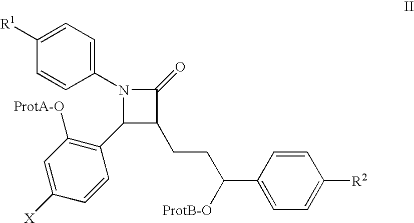

cyclizing a compound of formula IV

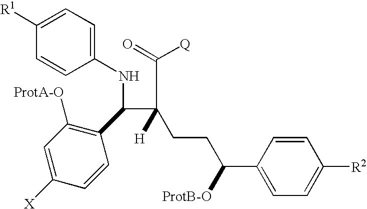

wherein Q is a chiral auxiliary attached at nitrogen. The chiral auxiliary may be chosen from single enantiomers of triphenyl glycol and cyclic and branched nitrogen-containing moieties possessing at least one chiral center. In one embodiment, the chiral auxiliary may be chosen from single enantiomers of cyclic and branched nitrogen-containing moieties attached at nitrogen. Examples of chiral auxiliaries include triphenyl glycol:

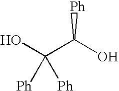

[see Braun and Galle, Synthesis 1996, 819-820], as well as the class of chiral nitrogen heterocycles:

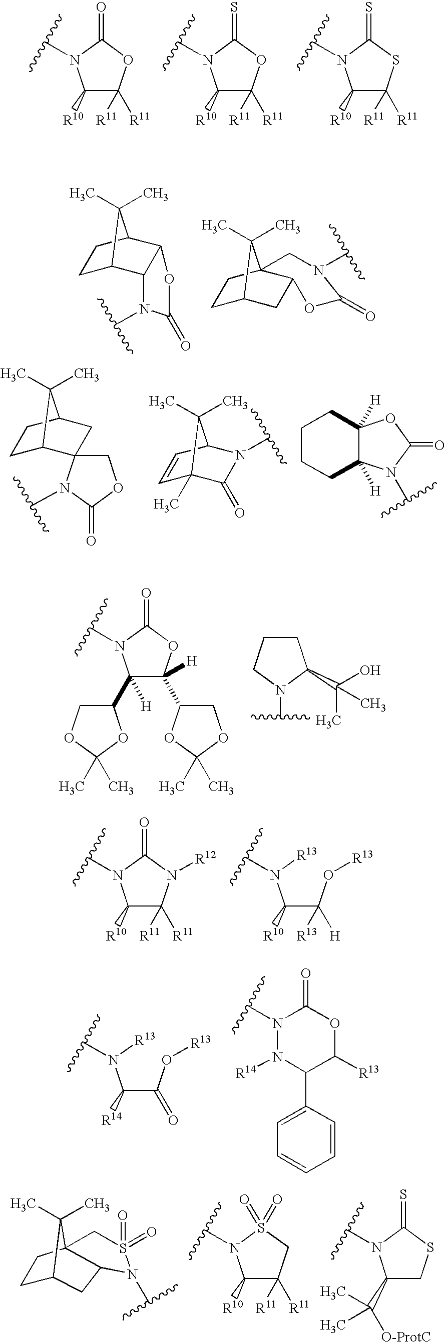

In these compounds, R¹⁰ is phenyl, benzyl, isopropyl, isobutyl or t-butyl; R¹¹ is hydrogen, methyl or ethyl; or R¹⁰ and R¹¹ together can form a cycle; R¹² is hydrogen, methyl or ethyl; R¹³ is hydrogen or methyl; R¹⁴ is methyl, benzyl, isopropyl, isobutyl or t-butyl; ProtC is methoxyoxymethyl (MOM), 2-(trimethylsilyl)ethoxymethyl (SEM), allyl or silyl [e.g. trimethylsilyl, t-butyldimethylsilyl, phenyldimethylsilyl]; and the wavy line indicates the bond by which the auxiliary is attached to the carbonyl of the parent. In one embodiment, IVa,

the chiral auxiliary is

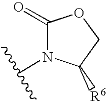

and R⁶ is phenyl or benzyl:

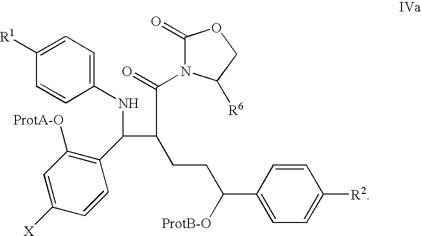

In one embodiment, in which ProtA-O— is methoxymethyl ether, allyl ether, t-butyl ether, silyl ether or benzyl ether and ProtB-O— is a silyl ether or tetrahydropyranyl ether, the cyclization is accomplished with N,O-bistrimethylsilylacetamide and a source of fluoride ion, such as tetrabutylammonium fluoride. The cyclization may also be carried out using a strong base, such as a metal hydride (e.g. sodium hydride, potassium hydride, lithium hydride).

The compound of formula IV

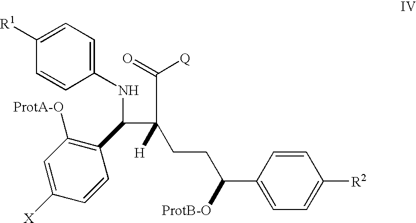

may be obtained by reacting a compound of formula V

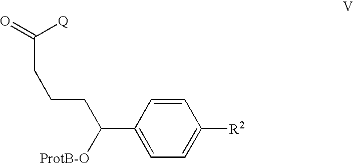

with a compound of formula VI

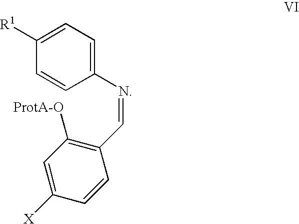

In one embodiment, compound of structure IVa

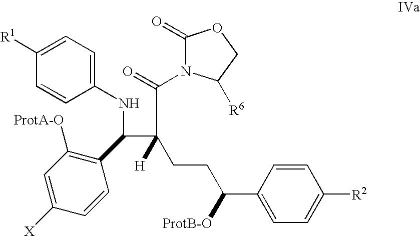

is produced by the sequential steps of
a. reacting a compound of formula Va

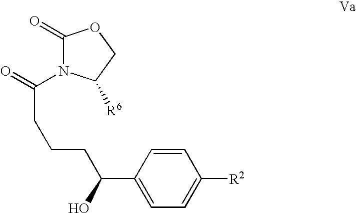

with a trialkylhalosilane in the presence of a base, such as an organic tertiary amine,
followed by
b. a Lewis acid, particularly a halide of a Group 3, 4, 13 or 14 metal, such as titanium tetrachloride;
followed by
c. a compound of formula VI

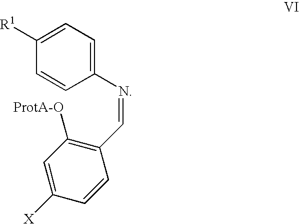

If the β-aminoacyloxazolinone component is protected (i.e. a compound of formula V in which ProtB-O is other than OH), “step a” can be omitted.

In another embodiment, a compound of formula

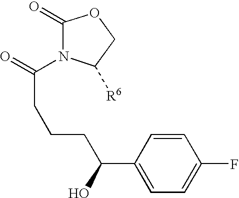

is reacted with trimethylchlorosilane in the presence of a tertiary amine to provide a silyl-protected benzyl alcohol, and the silyl-protected benzyl alcohol is reacted with titanium tetrachloride and an imine of formula

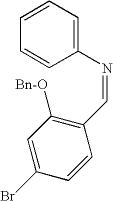

to provide a compound of formula

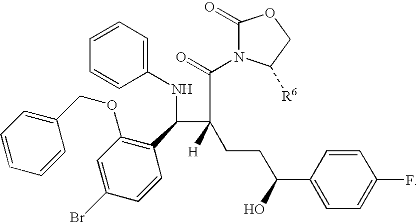

After the reaction of the silyl-protected benzyl alcohol with titanium tetrachloride and an imine, the product is isolated as a mixture in which the benzyl alcohol remains partly protected as the trimethylsilyl ether and partly deprotected to hydroxyl. The mixture can be converted entirely to the benzyl alcohol shown in the structure above by acid hydrolysis of the trimethylsilyl group and used in the next step or alternatively the mixture can be taken forward to the cyclization because the first part of the next step involves silylating the benzyl alcohol with N,O-bistrimethylsilylamide. Acid hydrolysis is preferred when the β-aminoacyloxazolinone will be purified by chromatography.

The compounds of formula V may be prepared by the process described in U.S. Pat. No. 6,627,757, in which Q is

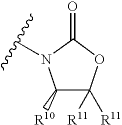

wherein R¹⁰ is phenyl and R¹¹ is hydrogen. Other chiral auxiliaries may be employed in the same fashion by replacing the N—H component

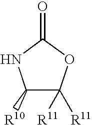

with any of the other appropriate Q groups described above.

The compounds of formula VI may be obtained by reacting a meta-substituted phenol with a source of formaldehyde followed by Schiff base formation with an aniline of formula

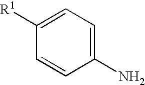

to produce a phenolic imine precursor to VI. The phenol is then protected under standard conditions appropriate for the chosen ProtA. For example, in the case in which ProtA is benzyl, the conditions are benzyl bromide and base. Sources of formaldehyde include paraformaldehyde, formaldehyde, trioxane and the like, all well known in the art. In the first step, the phenol reacts with formaldehyde in the presence of a magnesium salt, such as magnesium chloride, magnesium bromide or magnesium iodide, and a base. In the second step, the formylated phenol reacts with the aniline to provide the Schiff base VI.

Other routes to salicaldehydes may also be employed. Reaction of an appropriately substituted phenol in basic medium with formaldehyde (or chemical equivalent) will yield the corresponding salicylaldehyde. The intermediate, ortho-hydroxymethylphenol will be oxidized to the salicylaldehyde in situ. The reaction commonly employs ethyl magnesium bromide or magnesium methoxide (one equivalent) as the base, toluene as the solvent, paraformaldehyde (two or more equivalents) as the source of formaldehyde, and employs hexamethylphoramide (HMPA) or N,N,N′,N′-tetramethylethylenediamine (TMEDA). [See Casiraghi, G., et al., J. C. S. Perkin I, 1978, 318-321.] Alternatively the appropriately substituted phenol may react with formaldehyde under aqueous basic conditions to form the substituted ortho-hydroxybenzyl alcohol [See: a) J. Leroy and C. Wakselman, J. Fluorine Chem., 40, 23-32 (1988); b) A. A. Moshfegh, et al., Helv. Chim. Acta., 65, 1229-1232 (1982)], and the resulting ortho-hydroxybenzyl alcohol can be converted to the salicylaldehyde by an oxidizing agent such as manganese (IV) dioxide in a solvent such as methylene chloride or chloroform [See R-G. Xie, et al., Synthetic Commun. 24, 53-58 (1994)].

An appropriately substituted phenol can be treated under acidic conditions with hexamethylenetetramine (HMTA) to prepare the salicyladehyde. This is well known as the Duff Reaction. [See Y. Suzuki, and H. Takahashi, Chem. Pharm. Bull., 31, 1751-1753 (1983)]. The Duff reaction commonly employs acids such as acetic acid, boric acid, methanesulfonic acid, or trifluoromethanesulfonic acid. The source of formaldehyde commonly used is hexamethylenetetramine.

One may also employ the Reimer-Tiemann reaction, in which an appropriately substituted phenol will react under basic conditions with chloroform to yield a substituted salicylaldehyde. [See Cragoe, E. J, Schultz, E. M., U.S. Pat. No. 3,794,734 (1974)].

The formylation of the dilithium salt of a phenol with a formamide [see Talley and Evans, J. Org. Chem. 49, 5267-5269 (1984)] also provides salicaldehydes. The disclosures of all the foregoing salicaldehyde syntheses are incorporated herein by reference.

The compounds of formula III

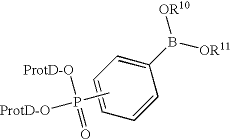

may be prepared according to the methods described below.

Also within the scope of the invention are compounds useful as intermediates in the processes described herein. The first of these is the class of intermediates of formula

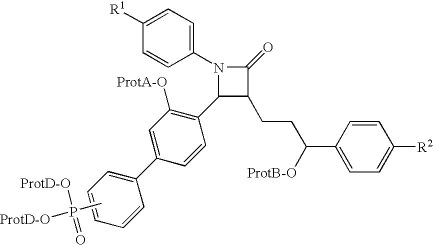

Specific embodiments of such intermediates include:

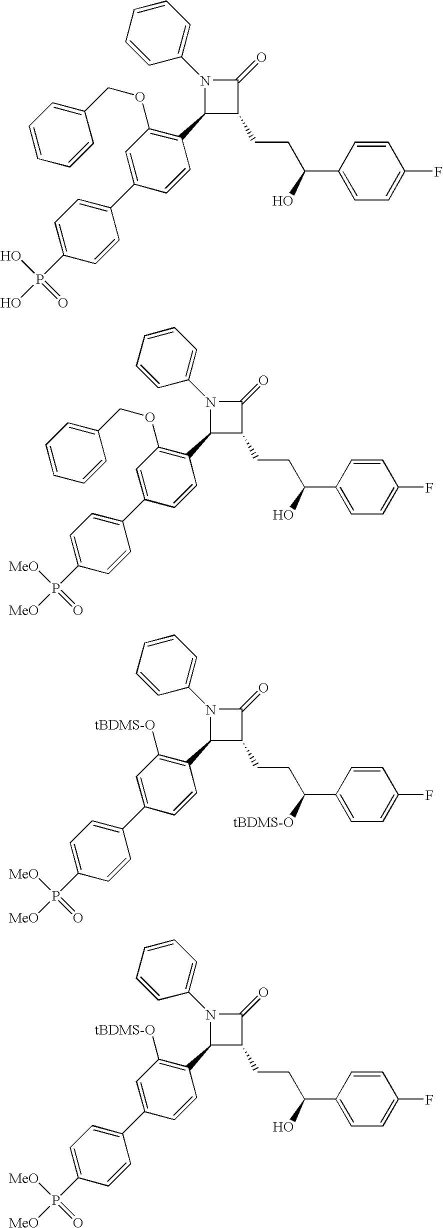

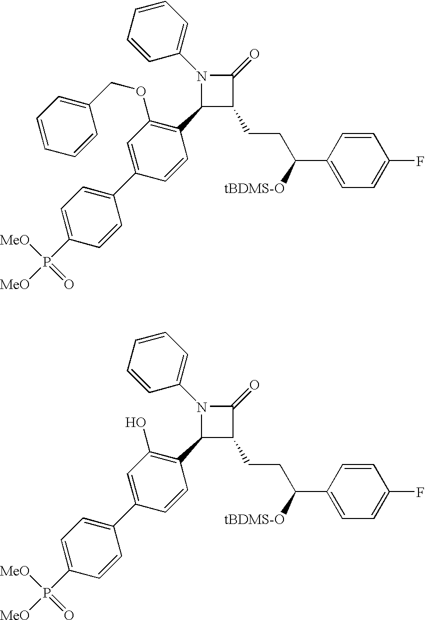

and the corresponding compounds in the 3-BPA series. As used herein, and as would be understood by the person of skill in the art, the recitation of “a compound” is intended to include salts, solvates and inclusion complexes of that compound. Thus, for example, a claim to a phosphonic acid such as the first example above would include the free acid and salts of the acid. Base addition salts for the acids of the present invention include metallic salts made from aluminum, calcium, lithium, magnesium, potassium, sodium and zinc or organic salts made from dicyclohexylamine, lysine, N,N′-dibenzylethylenediamine, chloroprocaine, choline, diethanolamine, ethylenediamine, meglumine (N-methylglucamine) and procaine.

A second novel class of compounds useful as intermediates in the processes described herein is the imines of formula VI

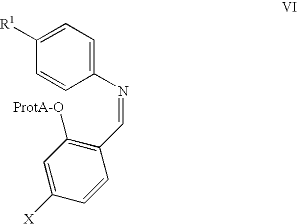

When ProtA- is benzyl, X is bromine and R¹ is H, the compound is solid and greater than 95% pure.

A third novel class of compounds useful as intermediates in the processes described herein are the Suzuki precursors of formula

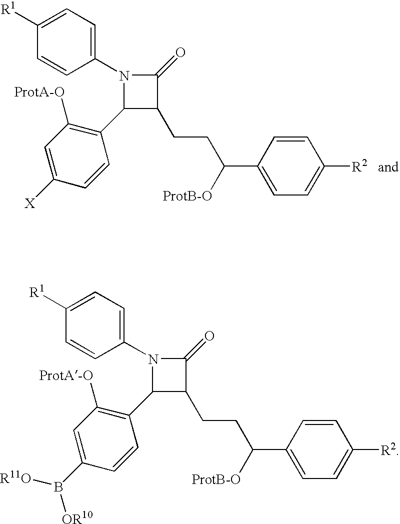

Examples of such include:

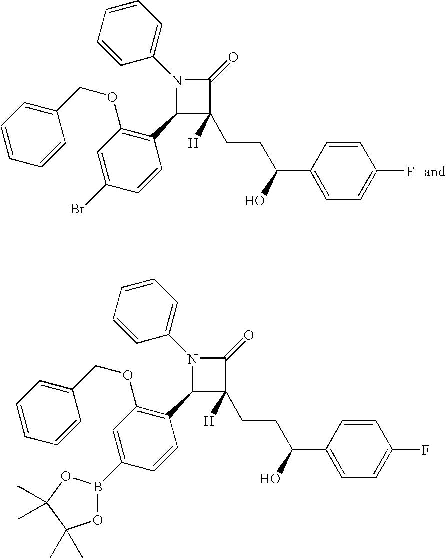

A fourth novel class of compounds useful as intermediates in the processes described herein are the precursors to the β-lactam of formula

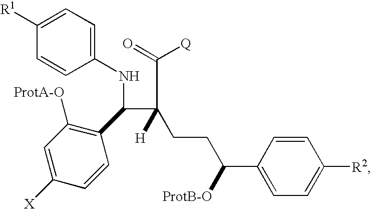

for example

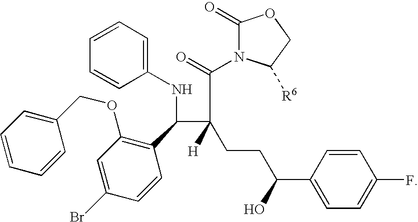

Exemplary processes that fall within the scope of the invention are illustrated in the schemes below. These schemes also illustrate the interrelatedness of the processes and intermediates. Schemes 5, 6 and 7 show alternate routes to 4-BPA.

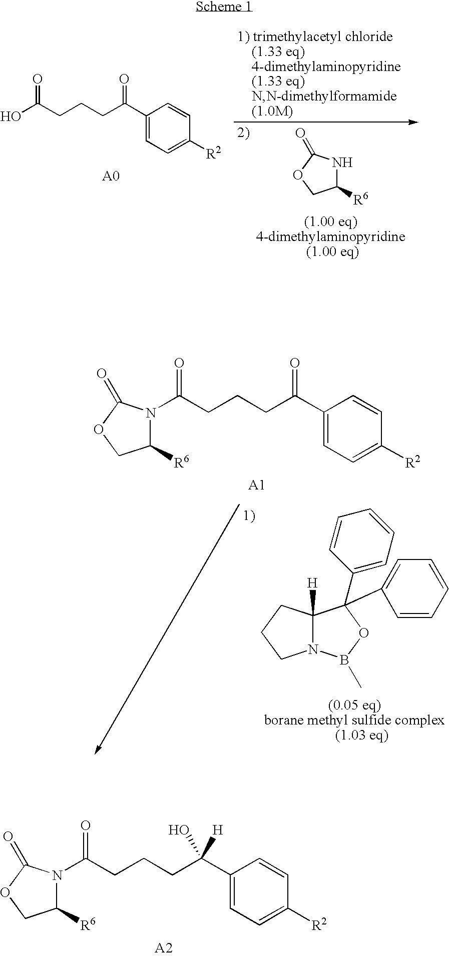

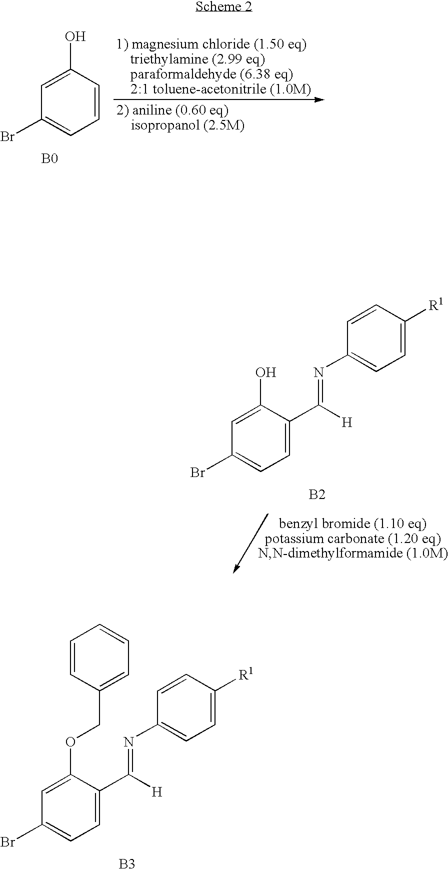

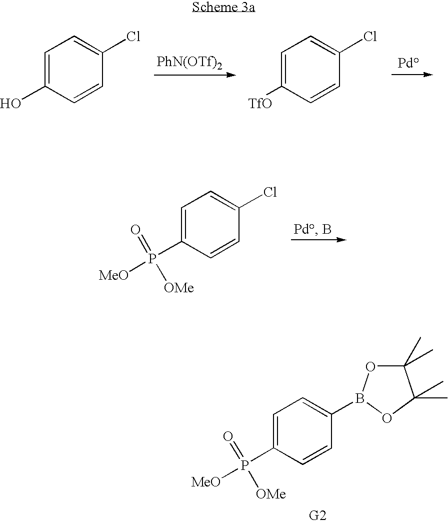

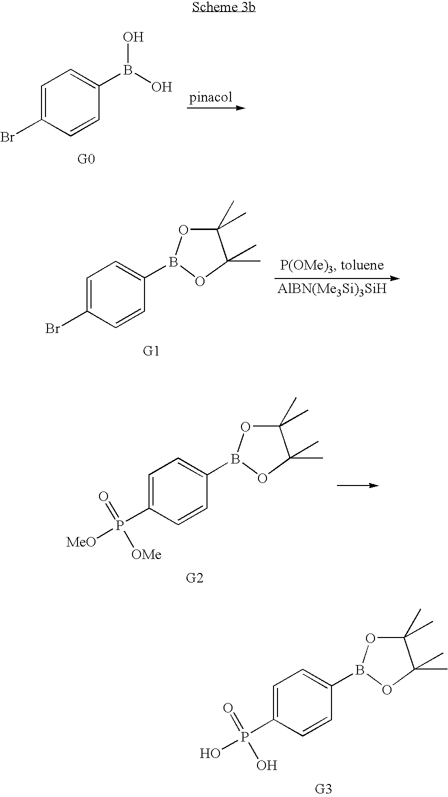

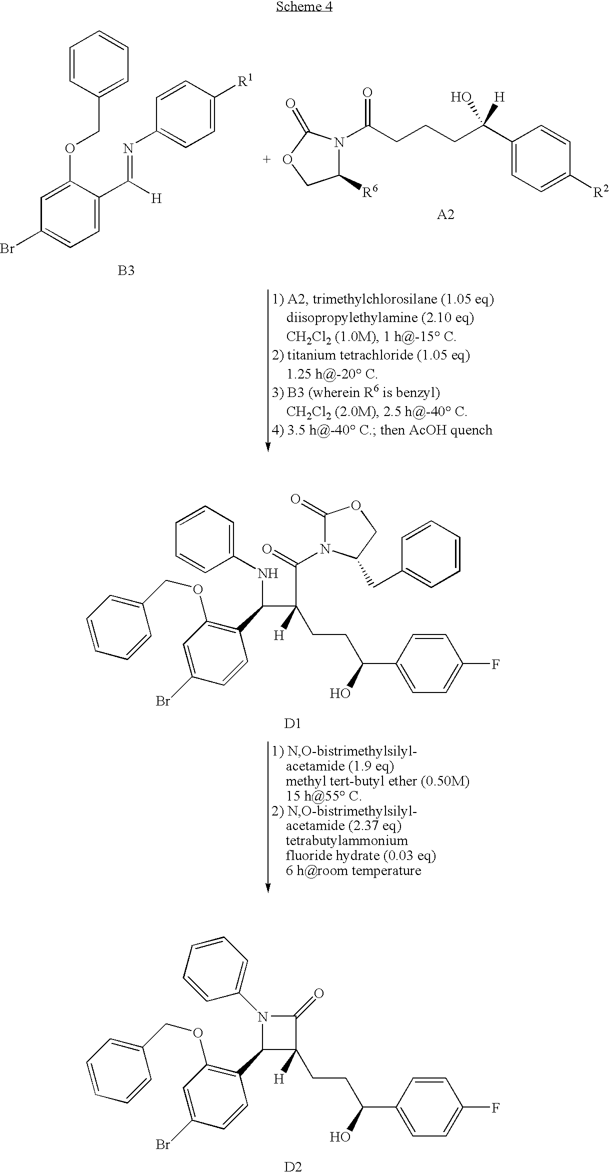

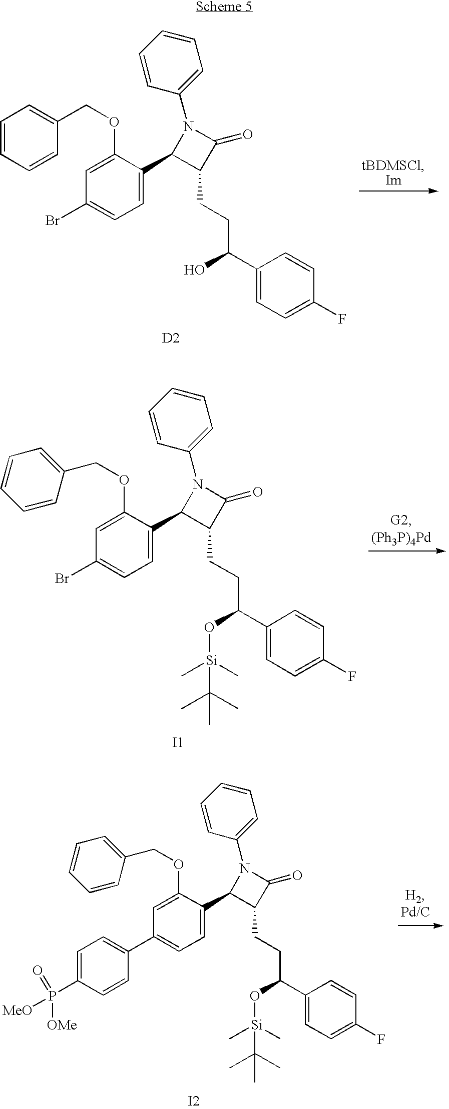

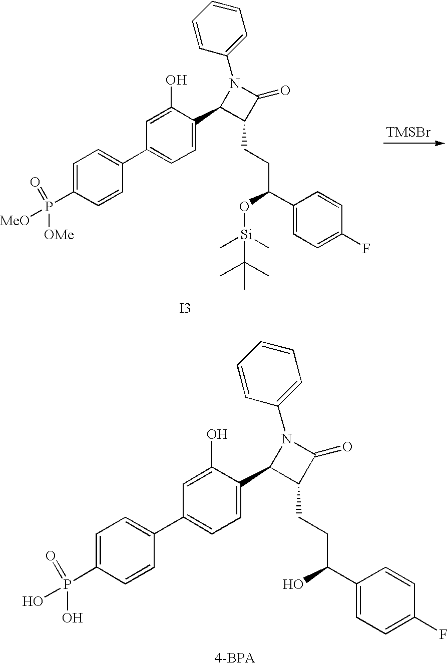

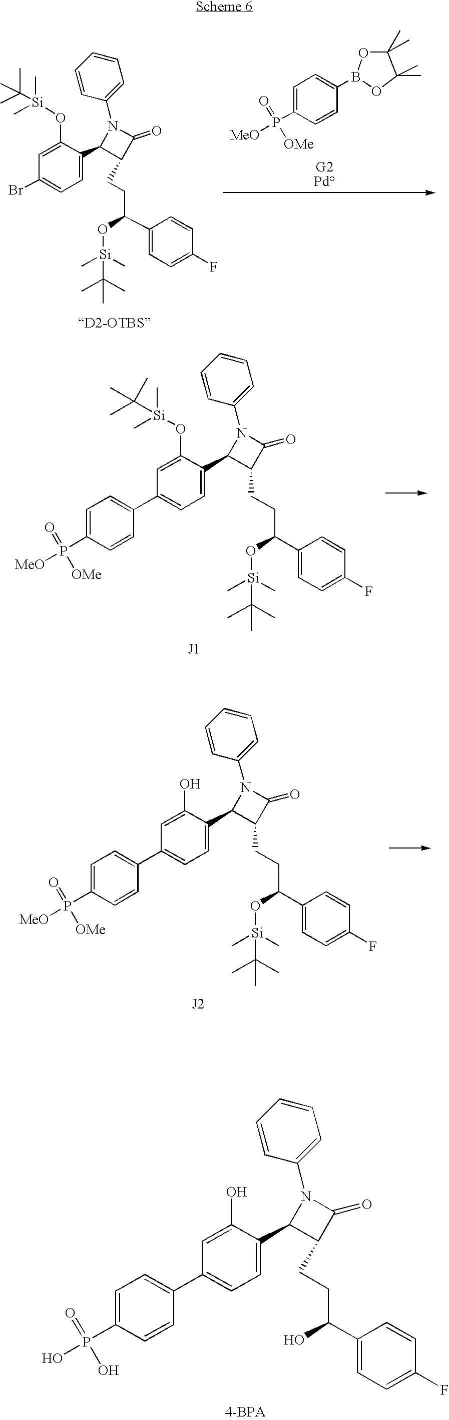

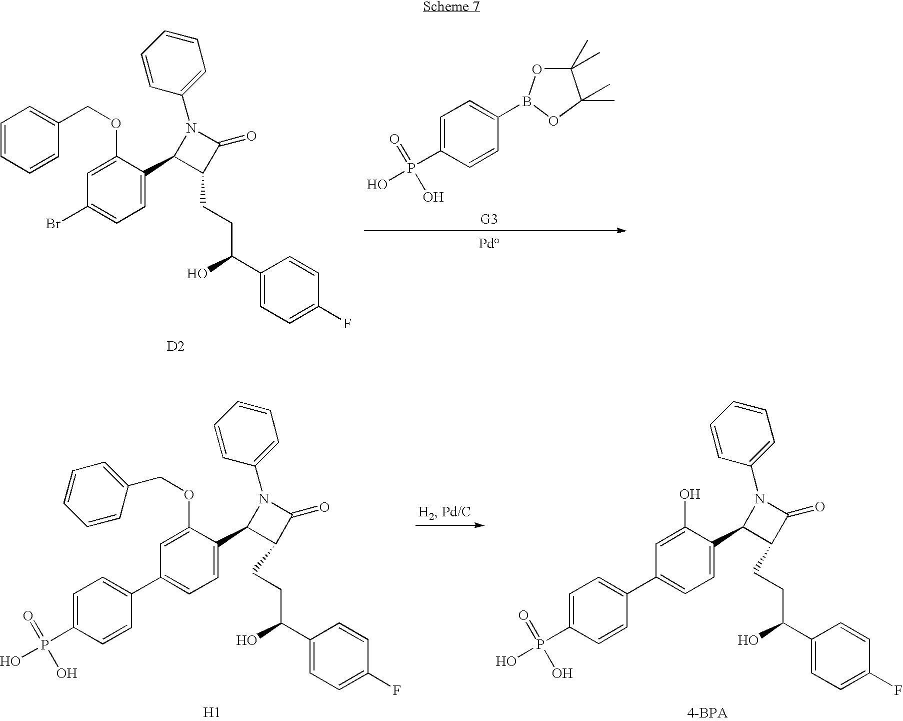

### Step 1. Preparation of (4S)-4-benzyl-3-[5-(4-fluorophenyl)-5-oxopentanoyl]-1,3-oxazolidin-2-one (A1)

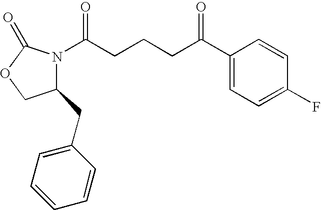

5-(4-Fluorophenyl)-5-oxopentanoic acid (372.0 g, 1.77 mol) and 4-dimethylamino-pyridine (286.9 g, 2.35 mol) were dissolved in N,N-dimethylformamide (1770 mL, 1.0 M) to afford a copious white precipitate suspended in solution. The reaction was cooled to 6° C. (ice/water bath), trimethylacetyl chloride (290 mL, 2.35 mol) was added quickly drop-wise over 17 min to afford a pale yellow mixture. The rate of addition was controlled in order to keep the temperature below 8.5° C. The mixture was stirred for 1 h at 9° C. (ice/water bath) then for 2 h at 20° C. (colorless solution with copious white thick precipitate). The mixture was charged with (S)-benzyl-2-oxazolidinone (313.5 g, 1.77 mol) and 4-dimethylaminopyridine (216.4 g, 1.77 mol) both as solids to afford a bright yellow colored suspension. The reaction was stirred at 27° C. for 3.3 h. The pale olive colored solution was poured into water (4300 mL) while stirring vigorously (an exotherm was detected to 39° C.), transferred with water (1000 mL) and stirred at room temperature for 2 h to afford a pale orange-brown solution with an off-white precipitate. The compound was filtered, transferred with water (2×300 mL), washed with water (400 mL) and air dried for 1.5 h to afford an off-white moist clumpy powder. The material was crystallized from isopropanol (2600 mL, 4.0 mL/g theoretical yield) by heating to near reflux to afford a dark golden yellow colored solution. The mixture was cooled slowly from 81° C. to 74° C. in 20 min, a seed crystal was added and crystals began to precipitate. The mixture was cooled slowly to room temperature over 11 h, cooled to 2° C. in an ice/water bath and stirred for 3 h. The crystals were filtered, transferred with cold mother liquor (350 mL), washed with cold isopropanol (2×350 mL), air dried and vacuum dried to constant weight to afford (4S)-4-benzyl-3-[5-(4-fluorophenyl)-5-oxopentanoyl]-1,3-oxazolidin-2-one (A1) (510.6 g, 78% yield) as a white crystalline solid; m.p. 113.4±1.2° C.; R_{f} 0.37 (1:2 ethyl acetate-hexane); HPLC purity 99.7 A % (96.4 A % by NMR); ¹H NMR (300 MHz, CDCl₃) δ 8.03-7.98 (m, 2H), 7.37-7.19 (m, 5H), 7.14 (t, J=8.7 Hz, 2H), 4.72-4.64 (m, 1H), 4.25-4.15 (m, 2H), 3.32 (dd, J=13.3, 3.4 Hz, 1H), 3.12-3.01 (m, 4H), 2.78 (dd, J=13.3, 9.6 Hz, 1H), 2.15 (quint., J=7.2 Hz, 2H) ppm.

In the synthesis of (4S)-4-benzyl-3-[5-(4-fluorophenyl)-5-oxopentanoyl]-1,3-oxazolidin-2-one (A1), two side products are formed:

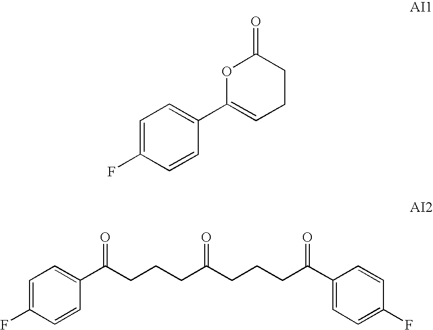

The first of these, AI1, can be reduced with hydrogen in the presence of a chiral catalyst to produce AI4

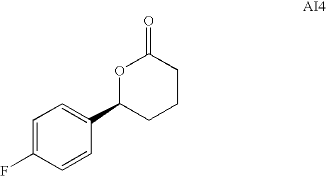

which can be utilized in the synthesis of D2 using the procedure described in PCT WO2004 099132. Although AI1 and AI2 were isolated by chromatography from the reaction described above, if one wishes to make AI1 directly, one can react 5-(4-fluorophenyl)-5-oxopentanoic acid with oxalyl chloride. The second by-product, AI2, if not removed, is subsequently reduced to AI3

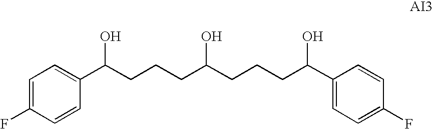

in the following step. It then co-crystallizes with A2 from toluene/alkane solvents and remains an impurity in A2. It can be removed from A2 by crystallization from isopropanol/alkane. The analytical assessment of the products is by TLC or HPLC with the following results:
A0—R_{f} 0.08 (1:2 ethyl acetate-hexane); HPLC R_{T} 3.7 min;
A1—R_{f} 0.37 (1:2 ethyl acetate-hexane); HPLC R_{T} 7.4 min;
A2—R_{f} 0.14 (1:2 ethyl acetate-hexane); HPLC R_{T} 6.5 min;
AI1—R_{f} 0.50 (1:2 ethyl acetate-hexane); HPLC R_{T} 5.5 min;
AI2—R_{f} 0.38 (1:2 ethyl acetate-hexane); HPLC R_{T} 7.6 min;
AI3—R_{f} 0.43 (2:1 ethyl acetate-hexane); HPLC R_{T} 5.4 min.
HPLC on Waters Xterra® MS C₁₈ (3.0×150 mm), 5 μm at 35° C.

| | |
|---|---|
| Mobile Phase (A): | 0.1% Formic Acid in Water (HPLC grade) |
| Mobile Phase (B): | Acetonitrile (HPLC grade) |
| Gradient Program: | 25% B - initial conditions |
| | 25% to 100% B - 11 min |
| | 100% to 25% B - 0.4 min |
| | 25% B - 3.6 min (flow increase to 1.75 mL/min) |
| Detection: | 254 nm |
| Flow Rate: | 1.0 mL/min |
| Run Time: | 15 min |

AI1 6-(4-fluorophenyl)-3,4-dihydro-2H-pyran-2-one. ¹H NMR (CDCl₃/300 MHz) 7.54 (dd, 2H, J=5.1, 9.0 Hz), 7.01 (dd, 2H, J=9.0, 9.0 Hz), 5.72 (t, 1H, J=4.8 Hz), 2.68-2.63 (m, 2H), 2.51-2.47 (m, 2H). Mass spectrum, M+H=193.

AI2 1,9-bis(4-fluorophenyl)nonane-1,5,9-trione, mp 97.1±0.7° C. ¹H NMR (CDCl₃/300 MHz) 7.92 (dd, 4H, J=5.4, 9.0 Hz), 7.06 (dd, 4H, J=9.0, 9.0 Hz), 2.92 (t, 4H, J=6.9 Hz), 2.49 (t, 4H, J=6.9 Hz), 1.95 (sept, 4H, J=6.9 Hz). Mass spectrum, M+H=359.

AI3 (1S,9S)-1,9-bis(4-fluorophenyl)nonane-1,5,9-triol. ¹H NMR (CDCl₃/300 MHz) 7.24 (dd, 4H, J=5.4, 8.4 Hz), 6.98 (dd, 4H, J=8.4, 8.4 Hz), 4.60 (m, 2H), 3.52 (m, 1H), 3.20-2.60 (m, 2H), 1.80-1.20 (m, 10H). Mass spectrum, M+H=365.

### Step 2. Preparation of (4S)-4-benzyl-3-[(5S)-5-(4-fluorophenyl)-5-hydroxypentanoyl]-1,3-oxazolidin-2-one (A2)

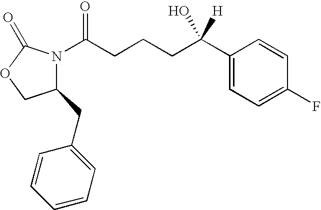

(4S)-4-Benzyl-3-[5-(4-fluorophenyl)-5-oxopentanoyl]-1,3-oxazolidin-2-one (A1) (500.0 g, 1.35 mol) was dissolved in dichloromethane (2700 mL, 0.5 M). The mixture was cooled to −4° C. (ice/brine bath), stirred for 40 min and charged with 1.0 M (R)-1-methyl-3,3-diphenyltetrahydro-3H-pyrrolo[1,2-c][1,3,2]oxazaborole in toluene (68 mL, 0.068 mol). After 10 min, borane-methyl sulfide complex (132 mL, 1.39 mol) was added drop-wise via addition funnel over 25 min (an exotherm was detected to −2.7° C.). The reaction was maintained between 0 and −6° C. with stirring for 3.0 h. The reaction was quenched by slow addition of methanol (275 mL, 6.79 mol) over 15 min (an exotherm was detected to 10° C.), 6% aqueous hydrogen peroxide (1150 mL, 2.02 mol) over 5 min and 1.0 M aqueous sulfuric acid (810 mL, 0.81 mol) over 15 min (an exotherm was detected to 17° C.) respectively via addition funnel. The reaction was stirred at room temperature for 60 min, poured into a separatory funnel, the organic layer was separated and the aqueous layer was extracted with dichloromethane (2000 mL). The first organic layer was washed with water (1500 mL) and brine (1500 mL). These aqueous layers were backed extracted with the second organic layer. The combined organic layers were partially concentrated, dried over sodium sulfate, filtered through Celite®, concentrated and crystallized from isopropanol-heptane (2000 mL, 1:1 isopropanol-heptane; 4.0 mL/g theoretical yield). The clear viscous residue was warmed to 42° C. (to make a homogeneous solution), cooled slowly to 35° C., held at this temperature for 12 h, cooled slowly to room temperature over 3 h, cooled to 0 to −5° C. (ice/brine bath) and stirred for 2 h. The crystals were filtered, transferred with cold mother liquor (250 mL), washed with cold 1:2 isopropanol-heptane (2×400 mL), air dried and vacuum dried to constant weight to afford (4S)-4-benzyl-3-[(5S)-5-(4-fluorophenyl)-5-hydroxypentanoyl]-1,3-oxazolidin-2-one (A2) (445.8 g, 89% yield) as a white crystalline solid; m.p. 75.4±0.6° C.; R_{f} 0.12 (1:2 ethyl acetate-hexane); HPLC purity 98.9 A %; ¹H NMR (300 MHz, CDCl₃) δ 7.37-7.24 (m, 5H), 7.19 (d, J=7.3 Hz, 2H), 7.02 (t, J=8.9 Hz, 2H), 4.72-4.61 (m, 2H), 4.21-4.13 (m, 2H), 3.27 (dd, J=13.2, 3.0 Hz, 1H), 2.99-2.94 (m, 2H), 2.74 (dd, J=13.2, 9.6 Hz, 1H), 2.27 (br s, 1H), 1.88-1.66 (m, 4H) ppm; [α]_{D}²³±72.9° (c 7.0, methanol).

### Step 3. Preparation of 5-bromo-2-[(E)-(phenylimino)methyl]phenol (B2)

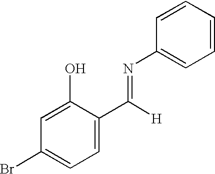

3-Bromophenol (498.5 g, 2.88 mol) was dissolved in a mixture of 2:1 toluene-acetonitrile (3000 mL, 0.96 M). To this solution was added triethylamine (1200 mL, 8.61 mol) via funnel. Magnesium chloride (412.7 g, 4.33 mol) was added in one portion as a solid (an exotherm was detected to 55° C.) to afford a bright yellow solution with copious white precipitate. Paraformaldehyde (345 g, 11.5 mol) was added as a suspension in acetonitrile (300 mL) while the temperature of the solution was 45° C. (an exotherm was detected to 78.6° C.). The temperature of the yellow-orange slurry was maintained at 80±3° C. for 1.5 h while the by-product (methanol) was distilled off (white precipitate was observed depositing in the distillation apparatus and reflux condensers). A second portion of paraformaldehyde (100 g, 3.33 mol) was added as a suspension in acetonitrile (200 mL). The mixture was heated for 2 h and another portion of paraformaldehyde (107 g, 3.56 mol) was added as a suspension in acetonitrile (200 mL). The mixture was stirred for 2.5 h at 80±4° C. After a total of 6 h and 6.4 equivalents total of paraformaldehyde had been added, the mixture was quenched with cold 2.5 N aqueous hydrochloric acid (6000 mL, 15 mol) added over 5 min. The mixture was stirred to room temperature for 60 min to afford a biphasic solution with a dull yellow top layer and dark orange bottom layer. The solution was diluted with 4:1 heptane-ethyl acetate (1000 mL), agitated and the layers separated. The aqueous layer was extracted with 4:1 heptane-ethyl acetate (2×1500 mL). Each organic layer was washed with the same portion of water (1800 mL) and brine (1800 mL). All the organic layers were combined, partially concentrated, dried over sodium sulfate, filtered through Celite® and concentrated to afford 2-hydroxy-4-bromobenzaldehyde as a dark golden-orange viscous oil; R_{f} 0.54 (1:4 ethyl acetate-hexane); HPLC purity 60 A %.

Crude 2-hydroxy-4-bromobenzaldehyde was dissolved in isopropanol (1000 mL, 1.26 mL/g theoretical yield, 2.5 M) and the mixture was heated to 75° C. Aniline (157 mL, 1.72 mol) was added to afford a bright orange solution and the mixture was left to cool slowly to room temperature (an exotherm was detected to 83° C. as imine crystallized from solution.) The mixture was stirred at room temperature for 12 h. The crystals were filtered, transferred with isopropanol (500 mL), washed with isopropanol (500 mL), air dried under a heavy stream of dry nitrogen gas and vacuum dried to constant weight to afford 5-bromo-2-[(E)-(phenylimino)methyl]phenol (B2) (347.4 g, 44% yield over two steps) as a bright yellow crystalline solid; m.p. 129.1±0.1° C.; R_{f} 0.65 (1:4 ethyl acetate-hexane); NMR purity >99 A %; ¹H NMR (300 MHz, CDCl₃) δ 8.59 (s, 1H), 7.47-7.40 (m, 2H), 7.33-7.22 (m, 5H), 7.08 (dd, J=8.2, 1.8 Hz, 1H), 1.57 (br s, 1H) ppm.

### Step 4. Preparation of N-{(1E)-[2-(benzyloxy)-4-bromophenyl]methylene}-N-phenylamine (B3)

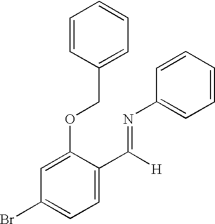

5-Bromo-2-[(E)-(phenylimino)methyl]phenol (B2) (310.9 g, 1.13 mol) was dissolved in anhydrous N,N-dimethylformamide (1100 mL, 1.0 M). Solid potassium carbonate (186.7 g, 1.35 mol) was added followed benzyl bromide (147.1 mL, 211.5 g, 1.24 mol) via syringe. The reaction was stirred under nitrogen for 4 h at room temperature and quenched with water (2000 mL) (an exotherm was detected to 40° C.). A yellow precipitate formed and the mixture was stirred for 1 h at room temperature. The solution was filtered and transferred with water (500 mL) and air dried under a heavy stream of dry nitrogen gas for 15 min. Crude solid was dissolved in isopropanol (1250 mL, 3.0 mL/g theoretical yield, 0.9 M) and the mixture was heated to 83° C. to afford a clear dark yellow solution which was cooled slowly to room temperature. The mixture was stirred at room temperature for 12 h. The crystals were filtered, transferred with cold isopropanol (250 mL), washed with cold isopropanol (250 mL), air dried under a heavy stream of dry nitrogen gas and vacuum dried to constant weight to afford N-{(1E)-[2-(1-benzyloxy)-4-bromophenyl]methylene}-N-phenylamine (B3) (375.2 g, 91% yield) as a light yellow crystalline solid; m.p. 100.2±0.2° C.; R_{f} 0.59 (1:4 ethyl acetate-hexane); NMR purity >99 A %; ¹H NMR (300 MHz, CDCl₃) δ 8.87 (s, 1H), 8.06 (d, J=8.2 Hz, 1H), 7.43-7.33 (m, 7H), 7.28-7.17 (m, 5H), 5.14 (s, 2H) ppm.

### Step 5. Preparation of (4S)-3-[(2R,5S)-2-{(S)-anilino[2-(benzyloxy)-4-bromophenyl]methyl}-5-(4-fluorophenyl)-5-hydroxypentanoyl]-4-benzyl-1,3-oxazolidin-2-one (D1)

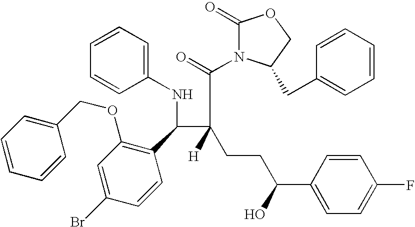

A 5-L three-necked flask was charged with (4S)-4-benzyl-3-[(5S)-5-(4-fluorophenyl)-5-hydroxypentanoyl]-1,3-oxazolidin-2-one (203.2 g, 0.547 mol) followed by addition of anhydrous dichloromethane (550 mL, 1.0 M) and N-ethyldiisopropylamine (200 mL, 148.4 g, 1.148 mol) via funnel. The reaction was cooled to −15° C. and trimethylchlorosilane (73.0 mL, 62.5 g, 0.575 mol) was added via cannula over 10 min (an exotherm was detected to −8° C.). The reaction was stirred for 1 h between −25° C. and −15° C. Titanium tetrachloride (63.0 mL, 109.0 g, 0.575 mol) was added drop-wise via addition funnel over 35 min to afford a deep reddish purple solution (an exotherm was detected to −10° C.). The mixture was stirred at −20±4° C. for 40 min, cooled to −40° C. and N-{(1E)-[2-(benzyloxy)-4-bromophenyl]methylene}-N-phenylamine (375.2 g, 1.024 mol) was added in dichloromethane (510 mL, 2.0 M) drop-wise slowly via addition funnel over 2.5 h. The reaction temperature was maintained between −45° C. and −31° C. The mixture was stirred for an additional 3.5 h, quenched by slow addition of glacial acetic acid (125 mL, 2.19 mol) over 15 min (the reaction temperature was maintained between −33° C. and −31° C.) and diluted with cold (10° C.) 15% aqueous dl-tartaric acid solution (2200 mL) (an exotherm was detected to 0° C.). This mixture was stirred to 17° C. over 2 h, diluted with dichloromethane (1000 mL), poured into a separatory funnel and the layers were separated. The organic layer was washed with 10% saturated brine solution (2000 mL) and brine (1000 mL). The aqueous layers were re-extracted sequentially with 1:1 ethyl acetate-heptane (2×1500 mL) and the combined organic layers were concentrated to afford a viscous reddish residue and copious yellow precipitate. The mixture was diluted with 1:4 dichloromethane-heptane (1000 mL), filtered and the solid was washed with 1:4 dichloromethane-heptane (3×500 mL). The filtrate was concentrated and the residue was diluted with dichloromethane (600 mL) and loaded onto silica gel (700 mL). The mixture was purified by pad filtration (300 mL silica gel, dichloromethane (300 mL) and 15% ethyl acetate-dichloromethane (4000 mL)) to afford (4S)-3-[(2R,5S)-2-{(S)-anilino[2-(benzyloxy)-4-bromophenyl]methyl}-5-(4-fluorophenyl)-5-hydroxypentanoyl]-4-benzyl-1,3-oxazolidin-2-one (D1) as a viscous, dark yellow, oil, which was used as-is in Step 4. ¹H NMR (300 MHz, CDCl₃) δ 7.50 (dd, J=8.2, 1.5 Hz, 2H), 7.39-7.30 (m, 3H), 7.26-6.98 (m, 12H), 6.94 (t, J=8.6 Hz, 2H), 6.62 (t, J=7.3 Hz, 1H), 6.52 (d, J=8.6 Hz, 2H), 5.13 (s, 2H), 5.06 (d, J=6.5 Hz, 1H), 4.73 (dd, J=13.8, 6.7 Hz, 1H), 4.64-4.57 (m, 1H), 4.49 (dd, J=7.3, 5.2 Hz, 1H), 4.12-4.04 (m, 2H), 3.01 (dd, J=13.4, 3.0 Hz, 1H), 2.39 (dd, J=13.4, 9.5 Hz, 1H), 1.84-1.51 (m, 6H) ppm.

### Step 6. Preparation of (3R,4S)-4-[2-(benzyloxy)-4-bromophenyl]-3-[(3S)-3-(4-fluorophenyl)-3-hydroxypropyl]-1-phenylazetidin-2-one (D2)

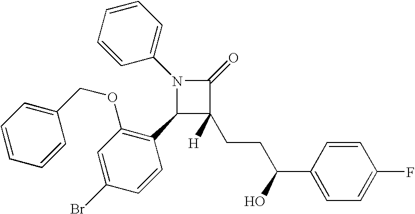

A 3-L three-necked flask was charged with semi-pure (4S)-3-[(2R,5S)-2-{(S)-anilino[2-(benzyloxy)-4-bromophenyl]methyl}-5-(4-fluorophenyl)-5-hydroxypentanoyl]-4-benzyl-1,3-oxazolidin-2-one (0.547 mol) in anhydrous tert-butyl methyl ether (1100 mL, 0.5 M) and N,O-bistrimethylsilylacetamide (250 mL, 1.012 mol, free of chlorotrimethylsilane) was added. The mixture was stirred at 55° C. for 15 h and then N,O-bistrimethylsilylacetamide (320 mL, 1.294 mol) was added followed by a catalytic amount of tetrabutylammonium fluoride trihydrate (4.62 g, 0.0177 mol) to afford a color change from bright yellow to pale golden yellow. The reaction was stirred at room temperature for 6 h and quenched with glacial acetic acid (1.0 mL, 0.018 mol). Hydrolysis of the silyl protecting groups is accomplished with 1.0 N aqueous hydrochloric acid (1100 mL) which was added drop-wise to avoid an exotherm (decomposition of the N,O-bistrimethylsilylacetamide with aqueous acid can be reactive). The bright yellow biphasic mixture was stirred for 1.5 h, poured into a separatory funnel, diluted with 1:1 ethyl acetate-heptane (1000 mL) and water (1000 mL), agitated, the layers were separated and the organic layer was washed with water (500 mL) and brine (500 mL). The organic layer can alternatively be washed with 5-25% sodium bisulfite, water (500 mL) and brine (500 mL). The two aqueous layers were back-extracted sequentially with one portion of 1:1 ethyl acetate-heptane (1000 mL) and the combined organic layers were concentrated. The residue was diluted with 1:1 heptane-dichloromethane (1000 mL), made into a slurry with silica gel (1000 mL) and purified by pad filtration (2000 mL silica gel, 10% (8000 mL), 20% (8000 mL), 30% (6000 mL) and 40% (4000 mL) ethyl acetate-hexane) to afford (3R,4S)-4-[2-(benzyloxy)-4-bromophenyl]-3-[(3S)-3-(4-fluorophenyl)-3-hydroxypropyl]-1-phenylazetidin-2-one (D2) (251.2 g, 82%) as a pale dull yellow foam; HPLC purity 89 A %; NMR purity 85 A %. A portion of the residue (124.2 g) was purified by crystallization from warm 8% water-methanol (500 mL, 4.0 mL/g, theoretical yield). The crystals were filtered, washed with cold 10% water-methanol (200 mL), air dried and vacuum dried to constant weight to afford (3R,4S)-4-[2-(benzyloxy)-4-bromophenyl]-3-[(3S)-3-(4-fluorophenyl)-3-hydroxypropyl]-1-phenylazetidin-2-one (D2) (85.9 g, 77% recovery based the amount of desired compound in the crude starting material) as white crystalline needles; m.p. 113±0.5° C.; R_{f} 0.32 (1:2 ethyl acetate-hexane); HPLC purity >99%; NMR purity >99%; ¹H NMR (300 MHz, CDCl₃) δ 7.41 (br s, 5H), 7.28-7.22 (m, 4H), 7.19-7.15 (m, 3H), 7.08-7.02 (m, 3H), 6.96 (t, J=8.7 Hz, 2H), 5.10 (dd, J=15.2, 11.2 Hz, 2H), 5.01 (d, J=2.4 Hz, 1H), 4.57-4.52 (m, 1H), 3.06-3.00 (m, 1H), 2.25 (d, J=3.8, 1H), 1.97-1.74 (m, 4H) ppm; [α]_{D}²³−12.3° (c 6.5, ethyl acetate).

### Alternate Route to (3R,48)-4-[2-(benzyloxy)-4-bromophenyl]-3-[(38)-3-(4-fluorophenyl)-3-hydroxypropyl]-1-phenylazetidin-2-one (D2)

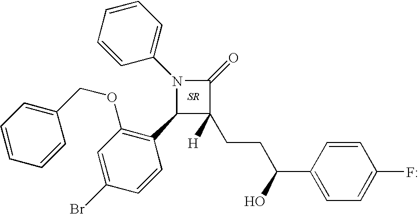

### Step 1A. Preparation of (4S-4-phenyl-3-[5-(4-fluorophenyl)-5-oxopentanoyl]-1,3-oxazolidin-2-one (the analog of compound A1 in which the 4-substituent is phenyl instead of benzyl, i.e. a precursor to Va in which R⁶ is phenyl)

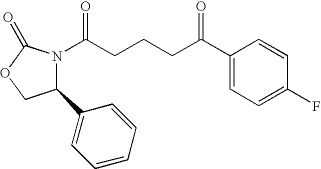

5-(4-Fluorophenyl)-5-oxopentanoic acid (21.02 g, 100.0 mmol) and 4 dimethylamino-pyridine (16.25 g, 133.0 mmol) were dissolved in N,N-dimethylformamide (100 mL, 1.0 M) to afford a copious white precipitate suspended in solution. The reaction was cooled to 2° C. (ice/water bath), and trimethylacetyl chloride (16.40 mL, 16.04 g, 133.0 mmol) was added drop-wise to afford a pale yellow mixture. The rate of addition was controlled in order to keep the temperature at or below 5° C. A heavy white precipitate was formed and the mixture was allowed to warm to room temperature and stirred for 1.5 h. The mixture was charged with (S)-(+)-4-phenyl-2-oxazolidinone (16.32 g, 100.0 mmol) and 4-dimethylaminopyridine (12.22 g, 100.0 mmol) both as solids to afford a yellow colored suspension. The reaction was stirred at 30° C.-35° C. for 2 h. An aliquot was removed for analysis by TLC and HPLC. The pale olive colored suspension was poured into water (400 mL) while stirring vigorously and cooling the mixture in an ice-brine bath, transferred with water (150 mL) and stirred with ice-cooling for 1.5 h to afford a solution with an off-white precipitate. The compound was filtered, transferred with water (2×25 mL), washed with water (50 mL) and air dried for 15 min to afford an off-white moist clumpy powder. The material was crystallized from isopropanol (58.0 mL; 1.6 mL/g theoretical yield) by heating to near reflux to afford a golden yellow colored solution. The solution was cooled slowly to room temperature over 12 h, a seed crystal was added and crystals began to precipitate. The mixture was cooled in an ice/water bath and stirred for 1 h. The crystals were filtered, transferred with cold isopropanol (2×10 mL), washed with cold isopropanol (25 mL), air dried and vacuum dried to constant weight to afford (4S)-4-phenyl-3-[5-(4-fluorophenyl)-5-oxopentanoyl]-1,3-oxazolidin-2-one (30.46 g, 85.7% yield) as a white crystalline solid; m.p. 91.0° C.; R_{f} 0.40 (1:2 ethyl acetate-hexane); HPLC R_{T} 7.02 min; HPLC purity 94%. ¹H NMR (300 MHz, CDCl₃) δ 7.93 (dd, J=5.4, 9.0 Hz, 2H), 7.28-7.42 (m, 5H), 7.10 (dd, J=8.5, 9.0 Hz, 2H), 5.43 (dd, J=3.7, 8.7 Hz, 1H), 4.70 (t, J=8.9 Hz, 1H), 4.28 (dd, J=3.7, 8.7 Hz, 1H), 3.05 (dt, J=1.2, 7.3 Hz, 2H), 2.97 (t, J=7.3, 2H), 2.05 (p, J=7.3 Hz, 2H), ppm.

### Step 2A. Preparation of (4S)-4-phenyl-3-[(5S)-5-(4-fluorophenyl)-5-hydroxypentanoyl]-1,3-oxazolidin-2-one (the analog of compound A2 in which the 4-substituent is phenyl instead of benzyl, i.e. a precursor to Va in which R⁶ is phenyl)

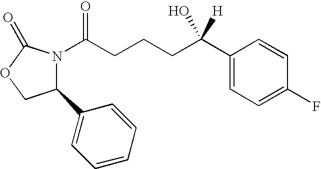

(4S)-4-Phenyl-3-[5-(4-fluorophenyl)-5-oxopentanoyl]-1,3-oxazolidin-2-one (28.43 g, 80.0 mmol) was dissolved in dichloromethane (160.0 mL; 0.5 M). The mixture was cooled to −10° C. (ice/brine bath), stirred for 10 min and charged with 1.0 M (R)-1-methyl-3,3-diphenyltetrahydro-3H-pyrrolo[1,2-c][1,3,2]oxazaborole in toluene (4.0 mL, 4.0 mmol), followed by dropwise addition of borane-methyl sulfide complex (7.80 mL, 6.26 g, 82.4 mmol). The addition rate was adjusted in order to keep the temperature at −8° C. The reaction temperature was maintained between −5 and −8° C. with stirring for 3.0 h. The reaction was quenched by slow addition of methanol (16.3 mL, 402.4 mmol), 6% aqueous hydrogen peroxide (68.2 mL, 120.0 mmol) and 1.0 M aqueous sulfuric acid (48.0 mL, 48 mmol) respectively, with ice-bath cooling. The cooling bath was then removed and the reaction was stirred at room temperature. After stirring at room temperature for 45 min, the mixture was poured into a separatory funnel, the organic layer was separated and the aqueous layer was extracted with dichloromethane (200 mL). The first organic layer was washed with water (125 mL) and brine (125 mL). The aqueous layers were backed extracted with the second organic layer. The combined organic layers were dried over sodium sulfate, filtered through Celite®, and concentrated to afford 31.9 g of a clear viscous film as crude product. This film was dissolved in 60 ml toluene at 50° C., cooled to room temperature, and crystallized over 12 h at −15° C. The white crystalline solid was filtered, transferred and washed with cold toluene (100 mL), air dried and vacuum dried to afford 24.45 g of a white solid. NMR analysis indicated the product to contain 6% toluene. The solid was again dissolved in toluene (50 mL) at 50° C. and hexane (50 mL) was added. The solution was cooled to room temperature with stirring and then stirred in an ice bath for 1 h. The white solid was filtered, transferred and washed with hexane (200 mL), air dried and vacuum dried to constant weight to afford (4S)-4-phenyl-3-[(5S)-5-(4-fluorophenyl)-5-hydroxypentanoyl]-1,3-oxazolidin-2-one (22.56 g, 79% yield) as a white crystalline solid; m.p. 39.7° C.; R_{f} 0.21 (2:3 ethyl acetate-hexane); HPLC R_{T} 6.09 min; HPLC purity 96.5%; ¹H NMR (300 MHz, CDCl₃) δ 7.15-7.42 (m, 7H), 7.00 (t, J=8.8 Hz, 2H), 5.40 (dd, J=3.7, 8.7 Hz, 1H), 4.68 (t, J=8.8 Hz, 1H), 4.59-4.66 (m, 1H), 4.27 (dd, J=3.7, 9.1 Hz, 1H), 2.93 (dt, J=1.1, 6.2 Hz, 2H), 1.58-1.80 (m, 4H) ppm.

### Step 5A. Preparation of 3-[2-[(2-Benzyloxy-4-bromo-phenyl)-phenylamino-methyl]-5-(4-fluoro-phenyl)-5-hydroxy-pentanoyl]-4-phenyl-oxazolidin-2-one.

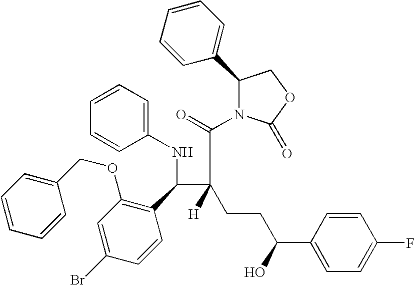

(4S)-4-phenyl-3-[(5S)-5-(4-fluorophenyl)-5-hydroxypentanoyl]-1,3-oxazolidin-2-one (21.4 g, 58.6 mmol) was dissolved in anhydrous dichloromethane (100 mL, 0.6 M) and cooled to −45° C. N-ethyldiisopropylamine (21.9 mL, 16.3 g, 125.8 mmol) was added slowly, followed by chlorotrimethylsilane (8.0 mL, 6.83 g, 62.9 mmol). The reaction was stirred for 1 h and the temperature was maintained between −20 and −30° C. Titanium tetrachloride (6.90 mL, 11.9 g, 62.9 mmol) was added drop-wise over 20 min to afford a deep reddish purple solution. The temperature was kept between −30 and −35° C. and stirring was continued for 45 min. The mixture was then cooled to −45° C. and a solution of N-{(1E)-[2-(benzyloxy)-4-bromophenyl]methylene}-N-phenylamine (B3) (37.3 g, 101.8 mmol) in dichloromethane (100 mL, 1.0 M) was added drop-wise over 30 min. The reaction temperature was maintained between −40° C. and −45° C. during addition. The mixture was stirred for 1.5 h between −40° C. and −45° C. An aliquot was removed for analysis by TLC and HPLC. The reaction was quenched by slow addition of glacial acetic acid (13.7 mL, 14.4 g, 240.0 mmol) over 10 min, followed by addition of cold (10° C.) 15% aqueous dl-tartaric acid solution (240.0 mL, 36.0 g, 240.0 mmol). The reaction mixture was warmed to −5° C. and was further allowed to warm up to room temperature after tartaric acid addition was completed. The mixture was stirred at room temperature over the next 1.5 h, diluted with dichloromethane (200 mL), poured into a separatory funnel and the layers were separated. The organic layer was washed with dilute brine solution (9:1 water/brine, 250 mL), then brine (100 mL). The aqueous layer was re-extracted sequentially with 1:1 ethyl acetate-hexane (200 mL, 150 mL). The combined organic layers were dried over Na₂SO₄ and concentrated to afford 59.4 g of an orange-red viscous oil. The crude product was dissolved in methanol (250 mL) and stored at −15° C. for 12 h. The resulting slurry was filtered to afford a white solid (27.7 g), suspended in methanol (150 mL) at 55° C., cooled in an ice-bath with stirring for 30 min to afford a white solid, filtered, transferred and washed with cold methanol (150 mL), air-dried and high-vacuum dried to afford 3-[2-[(2-Benzyloxy-4-bromo-phenyl)-phenylaminomethyl]-5-(4-fluoro-phenyl)-5-hydroxy-pentanoyl]-4-phenyl-oxazolidin-2-one (22.1 g, 51% yield) as a white powder; R_{f} 0.32 (1:1 ethyl acetate-Hexane); HPLC R_{T} 10.24 min; HPLC purity >99%; ¹H NMR (300 MHz, CDCl₃) δ 7.51 (dd, J=1.6, 8.3 Hz, 2H), 6.67-7.40 (m, 17H), 6.59 (tt, J=1.0, 7.4 Hz, 1H), 6.39 (dd, J=1.1, 8.6 Hz, 2H), 5.31-5.42 (m. 2H), 5.04-5.25 (m, 2H), 4.92 (dd, J=6.0, 9.5 Hz, 1H), 4.80 (dd, J=6.9, 13.3 Hz, 1H), 4.66 (t, J=8.6 Hz, 1H), 4.45-4.54 (m, 1H), 4.13 (dd, J=3.5, 8.8 Hz, 1H), 1.89 (d, J=3.4 Hz, 2H), 1.58-1.84 (m, 3H) ppm.

### Step 6A. Preparation of (3R,4S)-4-[2-(benzyloxy)-4-bromophenyl]-3-[(3S)-3-(4-fluorophenyl)-3-hydroxypropyl]-1-phenylazetidin-2-one (D2).

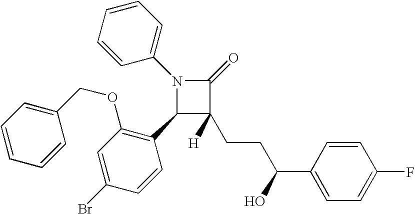

A 100 mL flask was charged with 3-[2-[(2-Benzyloxy-4-bromo-phenyl)-phenylamino-methyl]-5-(4-fluoro-phenyl)-5-hydroxy-pentanoyl]-4-phenyl-oxazolidin-2-one (1.45 g, 2.00 mmol) in anhydrous tert-butyl methyl ether (10 mL, 0.2 M) and N,O-bistrimethylsilylacetamide (1.0 mL, 4.00 mmol) was added. The clear solution was heated at reflux for 2 h with stirring. The heating bath was removed and a catalytic amount of tetrabutylammonium fluoride hydrate (0.050 g, 0.20 mmol) was added to afford a color change from colorless to pale yellow. Additional N,O-bistrimethylsilylacetamide (0.5 mL, 2.00 mmol) was added and the solution was stirred at room temperature for 16 h. The reaction was then cooled on ice and glacial acetic acid (0.01 mL, 0.20 mmol) was added, followed by 1.0 N aqueous hydrochloric acid (3.5 mL), which was added drop-wise to avoid an exotherm (decomposition of the N,O-bistrimethylsilylacetamide with aqueous acid can be reactive). The bright yellow biphasic mixture was stirred for 0.5 h, poured into a separatory funnel, diluted with 1:1 ethyl acetate-hexane (50 mL) and water (50 mL), agitated, the layers were separated and the organic layer was washed with water (50 mL) and brine (50 mL). The two aqueous layers were back-extracted sequentially with two portions of 1:1 ethyl acetate-hexane (2×30 mL) and the combined organic layers were dried over sodium sulfate and concentrated to afford 1.60 g yellow oil. The product was purified by column chromatography (ethyl acetate/hexane gradient 1:9 to 1:1) to afford (3R,4S)-4-[2-(benzyloxy)-4-bromophenyl]-3-[(3S)-3-(4-fluorophenyl)-3-hydroxypropyl]-1-phenylazetidin-2-one D2 (0.687 g, 61%) as a white solid (purity≧99% by LC-MS, R_{f}=0.30 [2:1 hexane/ethyl acetate], M(—OH⁻): 542.4 m/z); ¹H NMR (300 MHz, CDCl₃) δ 7.41 (br s, 5H), 7.28-7.22 (m, 4H), 7.19-7.15 (m, 3H), 7.08-7.02 (m, 3H), 6.96 (t, J=8.7 Hz, 2H), 5.10 (dd, J=15.2, 11.2 Hz, 2H), 5.01 (d, J=2.4 Hz, 1H), 4.57-4.52 (m, 1H), 3.06-3.00 (m, 1H), 2.25 (d, J=3.8, 1H), 1.97-1.74 (m, 4H) ppm; [α]_{D}²³−12.3° (c 6.5, ethyl acetate).

An alternative procedure used to crystallize D2 was as follows: The diastereomer ratio of D1 starting material was 79:21 [trans(total):cis(total)]. The crude D2 after work-up of the cyclization reaction, which totaled 135 g (Theory: 117 g of D2 diastereomers plus up to 37 g of cleaved benzyloxazolidinone) was heated in methanol (700 mL) to 65° C. Water (90 mL) was added dropwise to the stirred solution over 10 minutes. Seeds of diastereomerically pure D2 occasionally were added to the solution as it was cooled slowly to 47° C., held at 47° C. overnight, then finally cooled to room temperature over 5 hr. The solid was collected by filtration, then washed with ice-cold methanol/water (89:11) and dried under vacuum to give an off-white solid (D2, 54.0 g). No cis diastereomer could be detected by ¹H-NMR. The solid was heated to 50° C. in a mixture of methanol and isopropyl alcohol and charcoal was added. The solution was filtered and concentrated to dryness to give 43.9 g of white solid. This material was heated to 73° C. in isopropyl alcohol (228 mL) and a mixture of isopropyl alcohol/water (27:73, 104 mL) was added over 45 min. The solution was cooled to 65° C., seed crystals of diastereomerically pure D2 were added and the solution was allowed to cool slowly to room temperature. The solid was collected by filtration, washed with isopropyl alcohol/water (75:25, 80 mL) and dried under vacuum to give pure (3R,4S)-4-[2-(benzyloxy)-4-bromophenyl]-3-[(3S)-3-(4-fluorophenyl)-3-hydroxypropyl]-1-phenylazetidin-2-one (D2, 40.7 g, 44% yield from D1) as white needles, mp 113.9° C. The diastereomeric purity was determined to be 99.9% by chiral hplc analysis.

### Steps 7-9 for 3-BPA. Preparation of (4′-{(2S,3R)-3-[(3S)-3-(4-fluorophenyl)-3-hydroxypropyl]-4-oxo-1-phenylazetidin-2-yl}-3′-hydroxybiphenyl-3-yl)phosphonic acid (3-BPA)

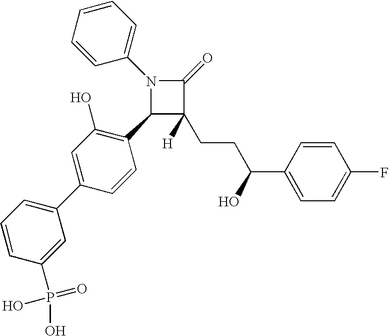

(3R,4S)-4-(4-Bromo-2-{[tert-butyl(dimethyl)silyl]oxy}phenyl)-3-[(3S)-3-{[tert-butyl(dimethyl)silyl]oxy}-3-(4-fluorophenyl)propyl]-1-phenylazetidin-2-one (0.080 g, 0.11 mmol), crude dimethyl[3-(4,4,5,5-tetramethyl-1,3,2-dioxaborolan-2-yl)phenyl]phosphonate (0.054 g total, 0.030 g calculated, 0.096 mmol) and aqueous 2 M potassium carbonate (0.12 mL, 0.24 mmol) were mixed in ethanol (1.0 mL) and toluene (3.0 mL). The solution was deoxygenated by bubbling nitrogen through the mixture for 5 min while stirring. Tetrakis(triphenylphosphine)palladium(0) (0.05 g) was added and the reaction was heated for 3 h at 70° C. under an atmosphere of nitrogen. The reaction was cooled to room temperature, diluted with ethyl acetate, washed with water and brine, dried over sodium sulfate and concentrated by rotary evaporation under reduced pressure. The product was purified by chromatography over silica gel using ethyl acetate-hexane (gradient: 10% ethyl acetate to 80%) to afford dimethyl (3′-{[tert-butyl(dimethyl)silyl]oxy}-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-3-yl)phosphonate as a colorless syrup (0.065 g, 84%). ¹H NMR (300 MHz, CDCl₃) δ 6.9-8.0 (m, 16H), 5.09 (d, J=2.2 Hz, 1H), 4.64 (d, J=6.1 Hz, 1H), 3.79 (d, J=2.4 Hz, 3H), 3.76 (d, J=2.4 Hz, 3H), 3.05-3.15 (m, 1H), 1.8-2.0 (m, 4H), 1.06 (s, 9H), 0.85 (s, 9H), 0.36 (s, 3H), 0.33 (s, 3H), 0.00 (s, 3H), −0.20 (s, 3H) ppm

Dimethyl (3′-{[tert-butyl(dimethyl)silyl]oxy}-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-3-yl)phosphonate (0.047 g, 0.058 mmol) was stirred at room temperature in dry methanol (2 mL) under a nitrogen atmosphere. Potassium fluoride (0.02 g, 0.34 mmol) was added and the reaction mixture was stirred for 30 min at room temperature. The solution was poured into ethyl acetate and washed successively with water (2×), and brine. The organic solution was dried over sodium sulfate, filtered and the solvent was removed by rotary evaporation under reduced pressure. Dimethyl (4′-{(2S,3R)-3-[(3S)-3-{[tert-butyl(dimethyl)silyl]oxy}-3-(4-fluorophenyl)propyl]-4-oxo-1 phenylazetidin-2-yl}-3′-hydroxybiphenyl-3-yl)phosphonate was obtained as a colorless glass (0.041 g, 100%) was used directly in the next reaction without further purification; MS [M−H]⁺ 688.

A solution of dimethyl (4′-{(2S,3R)-3-[(3S)-3-{[tert-butyl(dimethyl)silyl]oxy}-3-(4-fluorophenyl)propyl]-4-oxo-1-phenylazetidin-2-yl}-3′-hydroxybiphenyl-3-yl)phosphonate (0.041 g, 0.059 mmol) in dry dichloromethane (5 mL) under nitrogen was cooled in ice and bromotrimethylsilane (0.030 mL, 0.30 mmol) was dripped in over 5 min. The reaction mixture was stirred at room temperature for 3 h, then methanol (1 mL) was added and the reaction was partitioned between water and ethyl acetate. The organic solution was washed successively with water (2×) and brine. The organic solution was dried over sodium sulfate, filtered and the solvent was removed by rotary evaporation under reduced pressure. The residue was purified by reverse-phase HPLC (Polaris C18-A 10μ 250×21.2 mm column, 30% to 59% acetonitrile-0.1% trifluoroacetic acid in water) to afford (4′-{(2S,3R)-3-[(3S)-3-(4-fluorophenyl)-3-hydroxypropyl]-4-oxo-1-phenylazetidin-2-yl}-3′-hydroxybiphenyl-3-yl)phosphonic acid as a white powder (0.014 g, 44%); ¹H NMR (300 MHz, CD₃OD) δ 8.0 (d, J=13.6 Hz, 1H), 6.9-7.8 (m, 15H), 5.17 (d, J=2.1 Hz, 1H), 4.63 (d, J=5.2 Hz, 1H), 3.15-3.25 (m, 1H), 1.8-2.1 (m, 4H) ppm; MS [M−H]⁺ 546, [2M−H]⁺ 1093.

### Step 7. Preparation of dimethyl (3′-{[tert-butyl(dimethyl)silyl]oxy}-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-4-yl)phosphonate (J1)

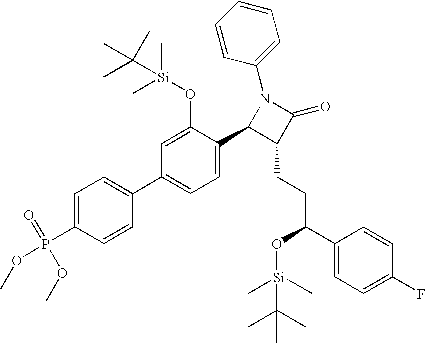

(3R,4S)-4-(4-Bromo-2-{[tert-butyl(dimethyl)silyl]oxy}phenyl)-3-[(3S)-3-{[tert-butyl(dimethyl)silyl]oxy}-3-(4-fluorophenyl)propyl]-1-phenylazetidin-2-one (5.10 g, 7.30 mmol), dimethyl[4-(4,4,5,5-tetramethyl-1,3,2-dioxaborolan-2-yl)phenyl]phosphonate (2.40 g, 7.70 mmol) and aqueous 2 M potassium carbonate (6.5 mL, 13 mmol) were mixed in ethanol (30 mL) and toluene (90 mL). The solution was deoxygenated by bubbling nitrogen through the mixture for 45 min while stirring. Tetrakis(triphenylphosphine)palladium(0) (0.38 g, 0.33 mmol) was added and the reaction was heated for 6 h at 75° C. under an atmosphere of nitrogen. The reaction was cooled to room temperature, diluted with ethyl acetate, washed with water and brine, dried over sodium sulfate and concentrated by rotary evaporation under reduced pressure to afford crude dimethyl (3′-{[tert-butyl(dimethyl)silyl]oxy}-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-4-yl)phosphonate.

### Step 8. Preparation of dimethyl (4′-{(2S,3R)-3-[(3S)-3-{[tert-butyl(dimethyl)silyl]oxy}-3-(4-fluorophenyl)propyl]-4-oxo-1-phenylazetidin-2-yl}-3′-hydroxybiphenyl-4-yl)phosphonate (J2)

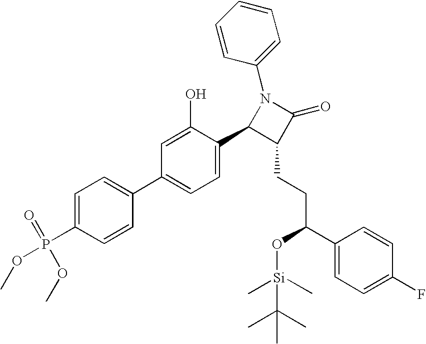

The crude dimethyl (3′-{[tert-butyl(dimethyl)silyl]oxy}-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-4-yl)phosphonate was stirred at room temperature in dry methanol (20 mL) under a nitrogen atmosphere. Potassium fluoride (0.84 g, 14.4 mmol) was added and the reaction mixture was stirred for 1 h at room temperature. The solution was poured into ethyl acetate and washed successively with water (3×), and brine. The organic solution was dried over sodium sulfate, filtered and the solvent was removed by rotary evaporation under reduced pressure. The product was purified by chromatography over silica gel using ethyl acetate-hexane (gradient: 40% ethyl acetate to 100%) to afford dimethyl (4′-{(2S,3R)-3-[(3S)-3-{[tert-butyl(dimethyl)silyl]oxy}-3-(4-fluorophenyl)propyl]-4-oxo-1-phenylazetidin-2-yl}-3′-hydroxybiphenyl-4-yl)phosphonate (3.10 g, 62% yield overall for two steps) as a white foam; ¹H NMR (300 MHz, CDCl₃) δ 9.36 (br s, 1H), 7.86 (dd, J=13.1, 8.6 Hz, 2H), 7.60 (dd, J=8.6, 4.1 Hz, 2H), 7.20-7.40 (m, 7H), 6.92-7.06 (m, 5H), 5.12 (d, J=2.4 Hz, 1H), 4.68 (dd, J=5.9, 4.2 Hz, 1H), 3.83 (d, J=11.4 Hz, 3H), 3.73 (d, J=11.3 Hz, 3H), 3.07-3.15 (m, 1H), 1.8-2.0 (m, 4H), 0.88 (s, 9H), 0.28 (s, 3H), −0.15 (s, 3H) ppm

### Step 9. Preparation of (4′-{(2S,3R)-3-[(3S)-3-(4-fluorophenyl)-3-hydroxypropyl]-4-oxo-1-phenylazetidin-2-yl}-3′-hydroxybiphenyl-4-yl)phosphonic acid (4-BPA)

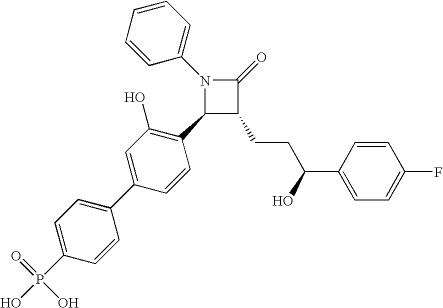

A solution of dimethyl (4′-{(2S,3R)-3-[(3S)-3-{[tert-butyl(dimethyl)silyl]oxy}-3-(4-fluorophenyl)propyl]-4-oxo-1-phenylazetidin-2-yl}-3′-hydroxybiphenyl-4-yl)phosphonate (0.26 g, 0.38 mmol) in dry dichloromethane (5 mL) under nitrogen was cooled in ice and bromotrimethylsilane (0.30 mL, 2.27 mmol) was drop-wise over 3 min. The reaction mixture was stirred at room temperature for 1 h, then methanol (1 mL) was added and the reaction was partitioned between water and ethyl acetate. The organic solution was washed successively with water (3×) and brine. The organic solution was dried over sodium sulfate, filtered and the solvent was removed by rotary evaporation under reduced pressure. The residue was purified by reverse-phase HPLC (Polaris C18-A 10μ 250×21.2 mm column, 30% to 59% acetonitrile-0.1% trifluoroacetic acid in water) to afford (4′-{(2S,3R)-3-[(3S)-3-(4-fluorophenyl)-3-hydroxypropyl]-4-oxo-1-phenylazetidin-2-yl}-3′-hydroxybiphenyl-4-yl)phosphonic acid (0.117 g, 56% yield) as a white powder; ¹H NMR (300 MHz, CD₃OD) δ 7.8 (dd, J=8.0, 13.0 Hz, 1H), 7.68 (dd, J=3.2, 8.0 Hz, 1H), 6.9-7.4 (m, 14H), 5.17 (d, J=2.1 Hz, 1H), 4.60-4.66 (m, 1H), 3.13-3.22 (m, 1H), 1.8-2.1 (m, 4H) ppm.

### Alternate Steps 7-9 for 4-BPA. (Shown in Scheme 5)

### Step Alt-7. Preparation of (3R,4S)-4-(4-Bromo-2-[benzyloxy]phenyl)-3-[(3S)-3-{[tert-butyl(dimethyl)silyl]oxy}-3-(4-fluorophenyl)propyl]-1-phenylazetidin-2-one (I1)

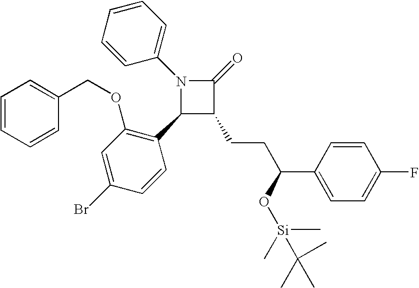

(3R,4S)-4-(4-Bromo-2-[benzyloxy]phenyl)-3-[(3S)-3-[hydroxy]-3-(4-fluorophenyl)propyl]-1-phenylazetidin-2-one (70.0 g, 124.9 mmol) was dissolved in dimethyl formamide (90 mL) and tert-butyl(dimethyl)silyl chloride (22.2 g, 147.4 mmol) and imidazole (10.6 g, 156.1 mmol) were added in succession at room temperature under a nitrogen atmosphere. The solution was heated at 50° C. for 19 h, then cooled to room temperature and diluted with ethyl acetate-hexane and mixed with water. The layers were separated, the organic layer was washed with water, brine and dried over sodium sulfate. The solution was filtered and the solvent was removed by rotary evaporation under reduced pressure to afford a white foam. The crude product was purified via pad filtration through silica gel and eluted with ethyl acetate-hexane to afford (3R,4S)-4-(4-bromo-2-[benzyloxy]phenyl)-3-[(3S)-3-{[tert-butyl(dimethyl)silyl]oxy}-3-(4-fluorophenyl)propyl]-1-phenylazetidin-2-one (83.4 g, 99% yield) as a white foam; ¹H NMR (300 MHz, CDCl₃) δ 7.00-7.50 (m, 16H), 6.90 (t, J=8.7 Hz, 1H), 5.13 (d, J=11.6 Hz, 2H), 5.06 (d, J=11.6 Hz, 1H), 4.98 (d, J=2.4 Hz, 1H), 4.52 (dd, J=5.4, 5.1 Hz, 1H), 2.99 (dt J=7.1, 2.3 Hz, 1H), 1.7-1.9 (m, 4H), 0.82 (s, 9H), 0.00 (s, 3H), −0.04 (s, 3H) ppm.

### Step Alt-8. Preparation of dimethyl (3′-[benzyloxy]-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-4-yl)phosphonate (I2)

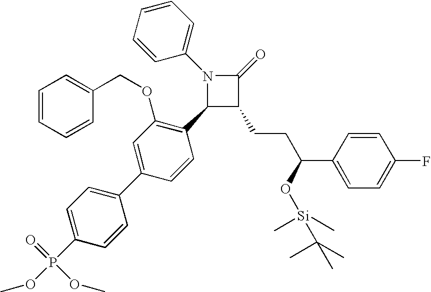

(3R,4S)-4-(4-Bromo-2-[benzyloxy]phenyl)-3-[(3S)-3-{[tert-butyl(dimethyl)silyl]oxy}-3-(4-fluorophenyl)propyl]-1-phenylazetidin-2-one (60.0 g, 88.9 mmol), dimethyl[4-(4,4,5,5-tetramethyl-1,3,2-dioxaborolan-2-yl)phenyl]phosphonate (34.0 g, 108.9 mmol) and aqueous 2 M potassium carbonate (92 mL, 184 mmol) were mixed in ethanol (150 mL) and toluene (450 mL). The solution was deoxygenated by bubbling nitrogen through the mixture for 1 h while stirring. Tetrakis(triphenylphosphine)palladium(0) (5.0 g, 4.3 mmol) was added and the reaction was heated for 4.5 h at 75° C. under an atmosphere of nitrogen. The reaction was cooled to room temperature and the layers were separated. The organic phase was washed with water and the combined aqueous phases were extracted with ethyl acetate. The combined organic phases were concentrated by rotary evaporation under reduced pressure. The residue was adsorbed onto a thick pad of silica gel and the product was eluted off using a gradient of ethyl acetate/hexane (1:9, 1:3, 1:1, 3:1) to give dimethyl (3′-[benzyloxy]-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-4-yl)phosphonate (61.8 g, 89% yield) as a tan foam; ¹H NMR (300 MHz, CDCl₃) δ 7.85 (dd, J=13.1, 8.5 Hz, 2H), 7.60 (dd, J=8.6, 3.9 Hz, 2H), 7.00-7.45 (m, 16H), 6.90 t, J=8.8 Hz, 1H), 5.24 (d, J=11.2 Hz, 2H), 5.17 (d, J=11.2 Hz, 1H), 5.10 (d, J=2.3 Hz, 1H), 4.55 (dd, J=5.6, 5.1 Hz, 1H), 3.80 (s, 3H), 3.77 (s, 3H), 3.07 (dt, J=7.0, 2.4 Hz, 1H), 1.75-1.92 (m, 4H), 0.83 (s, 9H), −0.03 (s, 3H), −0.19 (s, 3H) ppm.

### Step Alt-9. Preparation of dimethyl (3′-[hydroxy]-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-4-yl)phosphonate (I3)

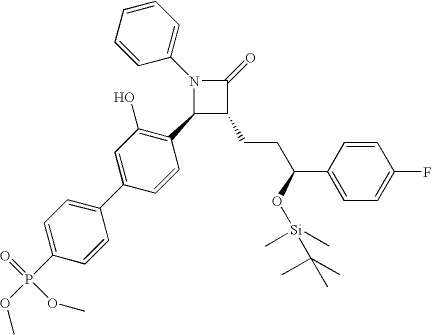

Dimethyl (3′-[benzyloxy]-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-4-yl)phosphonate (55.4 g, 70.1 mmol) was dissolved in 200-proof ethanol (100 mL) in a pressure vessel at room temperature. A slurry of 10% palladium on carbon, (12.0 g, water content: 52.76%) was added and hydrogen was bubbled through the solution for 5 min. The vessel was sealed and alternately pressurized with hydrogen gas (12 psi) and evacuated (3×). A pressure of 12 psi hydrogen gas was maintained overnight while the reaction mixture was rapidly stirred. The mixture was filtered through Celite® and the solvent was removed by rotary evaporation under reduced pressure to leave dimethyl (3′-[hydroxy]-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-4-yl)phosphonate (47.8 g, 99% yield) as a white foam; ¹H NMR (300 MHz, CDCl₃) δ 9.36 (br s, 1H), 7.86 (dd, J=13.1, 8.6 Hz, 2H), 7.60 (dd, J=8.6, 4.1 Hz, 2H), 7.20-7.40 (m, 7H), 6.92-7.06 (m, 5H), 5.12 (d, J=2.4 Hz, 1H), 4.68 (dd, J=5.9, 4.2 Hz, 1H), 3.83 (d, J=11.4 Hz, 3H), 3.73 (d, J=11.3 Hz, 3H), 3.07-3.15 (m, 1H), 1.8-2.0 (m, 4H), 0.88 (s, 9H), 0.28 (s, 3H), −0.15 (s, 3H) ppm.

### Step Alt-10. Preparation of (4′-{(2S,3R)-3-[(3S)-3-(4-Fluorophenyl)-3-hydroxypropyl]-4-oxo-1-phenylazetidin-2-yl}-3′-hydroxybiphenyl-4-yl)phosphonic acid (4-BPA)

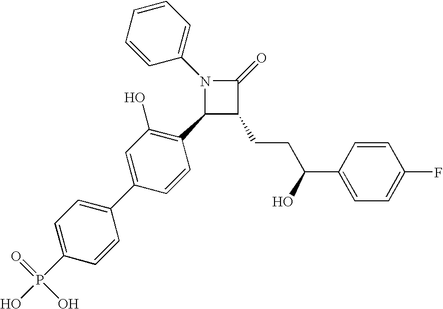

Dimethyl (3′-[hydroxy]-4′-{(2S,3R)-3-[(3S)-3-{[tert-butyl(dimethyl)silyl]oxy}-3-(4-fluorophenyl)propyl]-4-oxo-1-phenylazetidin-2-yl}biphenyl-4-yl)phosphonate (8.95 g, 13.0 mmol) in dry dichloromethane (60 mL) under nitrogen was cooled in ice and bromotrimethylsilane (10.0 mL, 75.8 mmol) was added drop-wise over 3 min. The reaction mixture was stirred at room temperature for 1 h, and concentrated to dryness by rotary evaporation under reduced pressure to leave a white foam (11.8 g). This residue was rapidly stirred in ethyl acetate (100 mL) and water (20 mL) for 20 min, and the layers were separated. The organic phase was washed with water (4×) and concentrated to dryness by rotary evaporation to give a white foam (8.7 g). A 2.5 g portion of this foam was purified by reverse-phase HPLC (Dynamax compression module, Polaris 10 C18-A 10μ 250×41.4 mm column, gradient running from 35% to 60% methanol-water) to afford (4′-{(2S,3R)-3-[(3S)-3-(4-fluorophenyl)-3-hydroxypropyl]-4-oxo-1-phenylazetidin-2-yl}-3′-hydroxybiphenyl-4-yl)phosphonic acid (1.45 g, which would represent 5.04 g if the entire material was purified, 71% yield) as a white powder; ¹H NMR (300 MHz, CD₃OD) δ 7.8 (dd, J=8.0, 13.0 Hz, 1H), 7.68 (dd, J=3.2, 8.0 Hz, 1H), 6.9-7.4 (m, 14H), 5.17 (d, J=2.1 Hz, 1H), 4.60-4.66 (m, 1H), 3.13-3.22 (m, 1H), 1.8-2.1 (m, 4H) ppm.

### Alternative Synthetic Route to 4-BPA (Shown in Scheme 7)

### Step 7-1. Preparation of (3′-(benzyloxy)-4′-{(2S,3R)-3-[(3S)-3-(4-fluorophenyl)-3-hydroxypropyl]-4-oxo-1-phenylazetidin-2-yl}biphenyl-4-yl)phosphonic acid (H1)

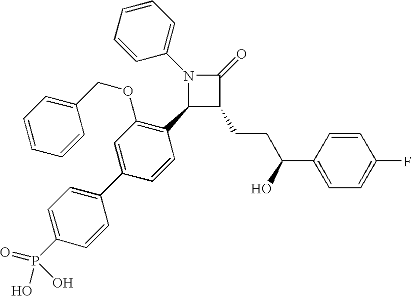

A 500-mL three-necked flask was charged with (3R,4S)-4-[2-(benzyloxy)-4-bromophenyl]-3-[(3S)-3-(4-fluorophenyl)-3-hydroxypropyl]-1-phenylazetidin-2-one (30.0 g, 53.5 mmol), [4-(4,4,5,5-tetramethyl-1,3,2-dioxaborolan-2-yl)phenyl]phosphonic acid (16.0 g, 56.3 mmol) and degassed 200-proof ethanol (54 mL, 1.0 M). The mixture was warmed to 45° C. to create a free-flowing slurry. Potassium phosphate (34.1 g, 160.6 mmol) was dissolved in degassed water (36 mL, 4.5 M) with heating and added to the reaction flask. trans-Bis(triphenylphosphine)palladium(II) dichloride (1.88 g, 2.68 mmol) was added as a slurry in 200-proof ethanol (2×9 mL) and the mixture was stirred at 45° C. while degassing with nitrogen gas bubbled directly into the solution for 10 min to displace oxygen. The solution turned a rusty color after 10 min upon reaching 72° C. and the mixture was heated to 80° C. which turns the solution homogeneous and dark brown. The reaction was stirred for 2 h at 80° C., cooled to 35° C., quenched with 2.5 N aqueous hydrochloric acid (300 mL) and ethyl acetate (150 mL), filtered through Celite®, and washed with ethyl acetate (150 mL). The mixture was agitated, the layers were separated and the organic layer was washed with 0.05 N aqueous hydrochloric acid (300 mL). The aqueous layers were back-extracted sequentially with ethyl acetate (300 mL) and the clear dark brown organic layers were combined and partially concentrated to 300 mL to reduce the volume of solvent but also to remove residual hydrochloric acid. Dicyclohexylamine (11.4 mL, 57.2 mmol) was added to the ethyl acetate solution to precipitate the phosphonate salt. The mixture was stirred vigorously while warming to 60° C. for 30 min, filtered warm and the filter cake was washed with warm ethyl acetate (2×100 mL). Air and vacuum dried to afford (3′-(benzyloxy)-4′-{(2S,3R)-3-[(3S)-3-(4-fluorophenyl)-3-hydroxypropyl]-4-oxo-1-phenylazetidin-2-yl}biphenyl-4-yl)phosphonic acid dicyclohexylammonium salt (H1.DCHA) (39.7 g, 91% yield) as a greyish-tan colored light solid; HPLC R_{T} 6.9 min; HPLC purity 90.9 A %; m.p. 235° C. (dec.); ¹H NMR (300 MHz, CD₃OD) δ 7.86 (dd, J=12.2, 8.1 Hz, 2H), 7.59 (dd, J=8.3, 2.8 Hz, 2H), 7.45-7.32 (m, 5H), 7.28-7.18 (m, 8H), 7.08-7.02 (m, 1H), 6.98 (t, J=8.7 Hz, 2H), 5.29 (d, J=11.9 Hz, 1H), 5.22 (d, J=11.9 Hz, 1H), 5.16 (d, J=2.3 Hz, 1H), 4.55-4.51 (m, 1H), 3.22-3.13 (m, 3H), 2.08-2.03 (m, 4H), 1.91-1.82 (m, 8H), 1.75-1.69 (M, 2H), 1.45-1.15 (m, 10H) ppm; LRMS [M−OH]⁺=620.6.

(3′-(Benzyloxy)-4′-{(2S,3R)-3-[(3S)-3-(4-fluorophenyl)-3-hydroxypropyl]-4-oxo-1-phenylazetidin-2-yl}biphenyl-4-yl)phosphonic acid dicyclohexylammonium salt (39.5 g, 48.2 mmol) was suspended in methanol (30 mL), 1.0 N aqueous hydrochloric acid (300 mL) and ethyl acetate (200 mL) were added. The mixture was stirred vigorously for 10 min, filtered through Celite®, and washed with ethyl acetate (100 mL). The layers were separated and the organic layer was washed with 0.05 N aqueous hydrochloric acid (2×200 mL). The aqueous layers were back-extracted sequentially with ethyl acetate (150 mL) and the organic layers were combined and concentrated. The material was dissolved in 200-proof ethanol (120 mL), treated with decolorizing charcoal (4.0 g) and Celite® (4.0 g), warmed to 50° C. for 30 min, cooled to room temperature, filtered through Celite®, washed with 200-proof ethanol (120 mL), and concentrated to afford (3′-(benzyloxy)-4′-{(2S,3R)-3-[(3S)-3-(4-fluorophenyl)-3-hydroxypropyl]-4-oxo-1-phenylazetidin-2-yl}biphenyl-4-yl)phosphonic acid (H1) (˜35.0 g, >100% yield due to trapped solvent) as a pale dark yellow-green foam which is used directly without further purification into the hydrogenolysis; HPLC R_{T} 6.7 min; ¹H NMR (300 MHz, CD₃OD) δ 7.85 (dd, J=12.7, 7.9 Hz, 2H), 7.68 (d, J=7.1 Hz, 2H), 7.45-7.33 (m, 5H), 7.26-7.16 (m, 9H), 7.06-7.00 (m, 1H), 6.97 (t, J=8.7 Hz, 2H), 5.28 (d, J=12.2 Hz, 1H), 5.21 (d, J=12.2 Hz, 1H), 5.15 (d, J=2.0 Hz, 1H), 4.54-4.51 (m, 1H), 3.18-3.12 (m, 1H), 1.96-1.80 (m, 4H) ppm.

### Step 7-2. Preparation of (4′-{(2S,3R)-3-[(3S)-3-(4-fluorophenyl)-3-hydroxypropyl]-4-oxo-1-phenylazetidin-2-yl}-3′-hydroxybiphenyl-4-yl)phosphonic acid (4-BPA)

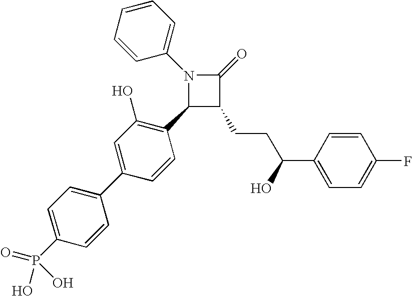

A 400-mL hydrogenation pressure flask was charged with (3′-(benzyloxy)-4′-{(2S,3R)-3-[(3S)-3-(4-fluorophenyl)-3-hydroxypropyl]-4-oxo-1-phenylazetidin-2-yl}biphenyl-4-yl)phosphonic acid (H1) (9.2 g, 14.4 mmol) in 200-proof ethanol (72 mL; 0.2 M). Wet 10% palladium on carbon (57.76% water by weight, 3.63 g, 1.44 mmol Pd) was added as a solid, the flask was pressurized to 14 psi with hydrogen gas and purged (10 cycles) and the black solution was stirred vigorously at 14 psi. The reaction was complete after 30 h, the pressure was released and the solution was purged with nitrogen gas for 15 min. The mixture was filtered through Celite® under a blanket of nitrogen gas, washed with warm (60° C.) 200-proof ethanol (100 mL), and concentrated in vacuo to afford (4′-{(2S,3R)-3-[(3S)-3-(4-fluorophenyl)-3-hydroxypropyl]-4-oxo-1-phenylazetidin-2-yl}-3′-hydroxybiphenyl-4-yl)phosphonic acid (4-BPA) (7.1 g, 90% yield) as an off white hard foam; HPLC R_{T} 5.0 min; HPLC purity 94.3 A %.

### Synthesis of Other Intermediates

### Preparation of (3R,4S)-3-[(3S)-3-{[tert-butyl(dimethyl)silyl]oxy}-3-(4-fluorophenyl)propyl]-4-[2-{[tert-butyl(dimethyl)silyl]oxy}-4-(4,4,5,5-tetramethyl-1,3,2-dioxaborolan-2-yl)phenyl]-1-phenylazetidin-2-one

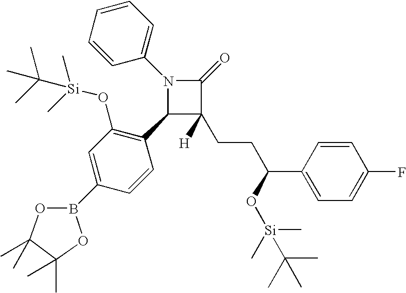

(3R,4S)-4-(4-Bromo-2-{[tert-butyl(dimethyl)silyl]oxy}phenyl)-3-[(3S)-3-{[tert-butyl(dimethyl)silyl]oxy}-3-(4-fluorophenyl)propyl]-1-phenylazetidin-2-one (0.42 g, 0.60 mmol) was dissolved in dioxane (15 mL) in a sealed tube. Bis(pinacolato)diboron (0.17 g, 0.66 mmol), potassium acetate (0.18 g, 1.83 mmol), and dichloro[1,1-bis(diphenylphosphino)ferrocene]palladium(II) dichloromethane adduct (14.6 mg, 0.018 mmol) were added and the reaction was degassed with argon and heated to 85° C. for 24 h. The mixture was cooled to room temperature diluted with 50 mL of 1:1 ethyl acetate-hexane, washed with 100 mL of 0.1 N hydrochloric acid and 2×100 mL of brine. The organic layers were collected, partially concentrated to half the volume, filtered through 10 g of silica gel, washed with 50 mL of ethyl acetate and concentrated in vacuo to afford (3R,4S)-3-[(3S)-3-{[tert-butyl(dimethyl)silyl]oxy}-3-(4-fluorophenyl)propyl]-4-[2-{[tert-butyl(dimethyl)silyl]oxy}-3-4-(4,4,5,5-tetramethyl-1,3,2-dioxaborolan-2-yl)phenyl]-1-phenylazetidin-2-one; ¹H NMR (300 MHz, CDCl₃) δ 7.35-7.18 (m, 9H), 7.02-6.96 (m, 1H), 6.95 (t, J=8.7 Hz, 2H), 5.11 (d, J=2.3 Hz, 1H), 4.63 (t, J=5.6 Hz, 1H), 3.06 (dt, J=7.4, 2.3 Hz, 1H), 1.96-1.79 (m, 4H), 1.31 (br s, 12H), 1.05 (s, 9H), 0.86 (s, 9H), 0.35 (s, 3H), 0.32 (s, 3H), 0.00 (s, 3H), −0.20 (s, 3H) ppm.

### Preparation of diethyl [4-(4,4,5,5-tetramethyl-1,3,2-dioxaborolan-2-yl)phenyl]phosphonate

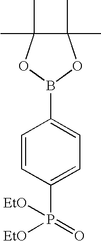

The Grignard reagent derived from the reaction of magnesium and para-dibromobenzene is reacted with diethyl chlorophosphate according to the procedure of Edder et al. [Org. Lett. 2003, 5, 1879-1882] to give diethyl 4-bromophenylphosphonate. Conversion of diethyl 4-bromophenylphosphonate to the corresponding pinacol boronate ester is accomplished by reaction with bis(pinicolato)diboron under the influence of palladium catalysis, essentially according to the procedure of Ishiyama et al. [J. Org. Chem. 1995, 60, 7508-7510]. (For additional references on the palladium catalyzed cross coupling see: A. Furstner, G. Seidel Org. Lett. 2002, 4, 541-543 and T. Ishiyama, M. Murata, T. Ahiko, N. Miyaura Org. Synth. 2000, 77, 176-185).

### Synthesis of dimethyl[4-(4,4,5,5-tetramethyl-1,3,2-dioxaborolan-2-yl)phenyl]phosphonate (Shown in Scheme 3b)

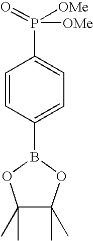

### Step 3b-1. Preparation of 4-Bromo-(4,4,5,5-tetramethyl-1,3,2-dioxaborolan-2-yl)benzene (G1)

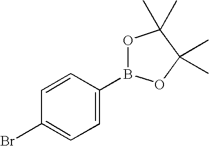

4-Bromophenyl boronic acid (52.6 g, 262 mmol) was suspended in acetonitrile (100 mL) at room temperature. Pinacol (29.5 g, 250 mmol) was added and the solution was stirred for 3 h at room temperature. The solvent was removed by rotary evaporation under reduced pressure and then under high vacuum to afford 4-bromo-(4,4,5,5-tetramethyl-1,3,2-dioxaborolan-2-yl)benzene (74.3 g, 105% yield) as an off-white solid that was used directly in the next reaction; ¹H NMR (300 MHz, CDCl₃) δ 7.66 (d, J=8.3 Hz, 2H), 7.50 (d, J=8.3 Hz, 2H), 1.34 (s, 12H) ppm.

### Step 3b-2. Preparation of dimethyl[4-(4,4,5,5-tetramethyl-1,3,2-dioxaborolan-2-yl)phenyl]phosphonate (G2)

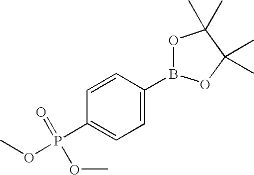

Crude 4-bromo-(4,4,5,5-tetramethyl-1,3,2-dioxaborolan-2-yl)benzene (74.3 g crude, 0.25 mol theory) was dissolved in toluene (300 mL, 0.82 M). To the solution was added trimethyl phosphite (94.0 mL, 0.797 mol) via funnel and the reaction was heated to 105° C. A solution of 1,1′-azobis(cyclohexanecarbonitrile) (9.8 g, 0.04 mol) and tris(trimethylsilyl) silane (97.2 mL, 0.315 mol) in toluene (200 mL) was added to the flask drop-wise over 4.5 hours at a rate of 1 mL/minute. Toluene was removed by distillation under vacuum, hexane (200 ml) was added and the reaction mixture was stirred at ambient temperature for 12 hours, then in an ice-water bath for 2 hours. The solid was filtered and washed with cold hexane (150 mL), air dried, then vacuum dried to constant weight to afford dimethyl[4-(4,4,5,5-tetramethyl-1,3,2-dioxaborolan-2-yl)phenyl]phosphonate (46.0 g, 56% yield) as a light cream-colored crystalline solid; mp 84.2±0.8° C.; R_{f} 0.29 (2:1 ethyl acetate-hexane); NMR purity >99 A %; ¹H NMR (300 MHz, CDCl₃) δ 7.89 (dd, J=8.2, 4.6 Hz, 2H), 7.81 (dd, J=13.2, 8.2 Hz, 2H), 3.75 (s, 3H), 3.72 (s, 3H), 1.34 (s, 12H) ppm; MS [M+H] 312, [2M+H] 625.

### Preparation of dimethyl[3-(4,4,5,5-tetramethyl-1,3,2-dioxaborolan-2-yl)phenyl]phosphonate

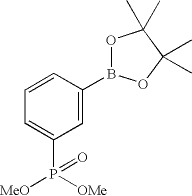

3-Chlorophenol (0.50 g, 3.89 mmol) was stirred at room temperature in dry dichloromethane (20 mL) under a nitrogen atmosphere. Phenyltrifluoromethanesulfonimide (1.80 g, 5.0 mmol), triethylamine (0.90 mL, 6.4 mmol) and 4-dimethylaminopyridine (0.10 g, 0.8 mmol) were added in succession and the reaction mixture was stirred 2 h at room temperature. The solution was poured into 0.5 N hydrochloric acid (20 mL) and extracted with ethyl acetate. The organic phase was washed successively with water, 10% aqueous sodium bicarbonate and brine. The organic solution was dried over sodium sulfate, filtered and the solvent was removed by rotary evaporation under reduced pressure. Pure 3-chlorophenyl trifluoromethanesulfonate was obtained as a colorless oil (0.92 g, 91%) by chromatography over silica gel using ethyl acetate-hexane (gradient: 5% to 50% ethyl acetate-hexane); ¹H NMR (300 MHz, CDCl₃) δ 7.16-7.50 (m) ppm.

This reaction was performed using a PersonalChemistry™ microwave instrument set at normal absorbance, fixed hold time and 30 sec pre-stirring. A 10-mL reaction vial was charged with 3-chlorophenyl trifluoromethanesulfonate (0.60 g, 2.30 mmol), dimethyl phosphite (0.42 mL, 4.58 mmol) and triethylamine (0.64 mL, 4.59 mmol) in toluene (4 mL). Nitrogen was bubbled through the stirred solution for 5 min, the tetrakis(triphenylphosphine)palladium(0) (0.1 g) was added, the solution was covered with a blanket of nitrogen and sealed. The reaction mixture was heated 11 min at 160° C., then cooled to room temperature and diluted with ethyl acetate. The yellow solution washed successively with water (3×) and brine. The organic solution was dried over sodium sulfate, filtered and the solvent was removed by rotary evaporation under reduced pressure. Pure dimethyl (3-chlorophenyl)phosphonate was obtained as a colorless oil (0.27 g, 57%) by chromatography over silica gel using ethyl acetate-hexane (gradient: 5% ethyl acetate to 100%). ¹H NMR (300 MHz, CDCl₃) δ 7.77 (br d, J=13.7 Hz, 1H), 7.68 (ddt, J=13.0, 7.5, 1.4 Hz, 1H), 7.53 (dquint., J=8.0, 1.1 Hz, 1H), 7.38-7.45 (m, 1H), 3.79 (s, 3H), 3.75 (s, 3H) ppm; MS [M+H]⁺ 221, [2M+H]⁺ 441.

Bis(dibenzylidineacetone)palladium(0) (0.10 g, 0.17 mmol and tricyclohexylphosphine (0.12 g, 0.43 mmol) were stirred 30 min in dry dioxane (1.0 mL) under an atmosphere of nitrogen at room temperature. Dimethyl (3-chlorophenyl)phosphonate (0.50 g, 2.26 mmol), bis(pinacolato)diboron (0.70 g, 0.27 mmol) and potassium acetate (0.30 g, 0.30 mmol) were mixed in dry dioxane (3.0 mL) at room temperature under a nitrogen atmosphere in a separate flask. A portion of the palladium catalyst solution (0.20 mL) was syringed into the flask containing the chlorophosphonate and this mixture was heated at 80° C. Additional 0.2 mL portions of the catalyst solution were syringed into the reaction mixture after 4 h and 8 h of heating at 80° C., then heating was continued overnight at 80° C. The reaction mixture was filtered through Celite® and the solvent was removed by rotary evaporation under reduced pressure. Chromatography over silica gel using ethyl acetate-hexane (gradient: 0% ethyl acetate to 80%) dimethyl[3-(4,4,5,5-tetramethyl-1,3,2-dioxaborolan-2-yl)phenyl]phosphonate as a colorless oil (0.41 g). ¹H NMR showed a 60:40 mixture of product plus recovered starting material. This mixture was used as is in the next reaction without further purification. ¹H NMR (300 MHz, CDCl₃) δ 8.22 (d, J=13.2 Hz, 1H), 7.95-8.00 (m, 1H), 7.88 (ddt, J=13.0, 7.5, 1.4 Hz, 1H), 7.43-7.50 (m, 1H), 3.76 (s, 3H), 3.73 (s, 3H) ppm; MS [M+H]⁺ 312, [2M+H]⁺ 625.

### Synthesis of dimethyl[4-(4,4,5,5-tetramethyl-1,3,2-dioxaborolan-2-yl)phenyl]phosphonate (G2) (Shown in Scheme 3a)

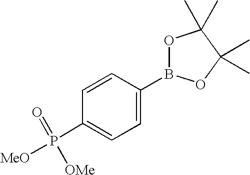

### Step 3a-1. Preparation of 4-Chlorophenyl trifluoromethanesulfonate (K1)

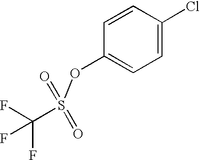

4-Chlorophenol (3.00 g, 23.3 mmol) was stirred at room temperature in dry dichloromethane (40 mL) under a nitrogen atmosphere. N-Phenyl-bis(trifluoromethanesulfonimide) (10.00 g, 28.0 mmol), triethylamine (5.1 mL, 36.5 mmol) and 4-dimethylaminopyridine (0.10 g, 0.8 mmol) were added in succession and the reaction mixture was stirred 3 h at room temperature. The solution was poured into 0.5 N aqueous hydrochloric acid (100 mL) and extracted with ethyl acetate. The organic phase was washed successively with water, 10% aqueous sodium bicarbonate and brine. The organic solution was dried over sodium sulfate, filtered and the solvent was removed by rotary evaporation under reduced pressure. Pure 4-chlorophenyl trifluoromethanesulfonate (5.65 g, 93% yield) was obtained as a colorless oil by chromatography over silica gel using ethyl acetate-hexane (gradient: 5% to 50% ethyl acetate-hexane); ¹H NMR (300 MHz, CDCl₃) δ 7.43 (d, J=9.1 Hz, 2H), 7.23 (d, J=9.1 Hz, 2H), ppm.

### Step 3a-2. Preparation of dimethyl (4-chlorophenyl)phosphonate (K2)

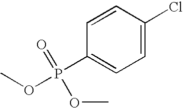

A reaction flask containing 4-chlorophenyl trifluoromethanesulfonate (1.50 g, 5.76 mmol), dimethyl phosphite (0.90 mL, 9.81 mmol) and triethylamine (1.60 mL, 11.4 mmol) in toluene (25 mL) was degassed by bubbling nitrogen through the stirred solution for 10 min. Tetrakis(triphenylphosphine)palladium(0) (0.1 g) was added and the reaction mixture was heated at reflux for 6 h, cooled to room temperature and diluted with ethyl acetate. The yellow solution was washed successively with water (2×) and brine. The organic solution was dried over sodium sulfate, filtered and the solvent was removed by rotary evaporation under reduced pressure. Pure dimethyl (4-chlorophenyl)phosphonate (1.01 g, 79% yield) was obtained as a colorless oil by chromatography over silica gel using ethyl acetate-hexane (gradient: 5% ethyl acetate to 100%); ¹H NMR (300 MHz, CDCl₃) δ 7.75 (dd, J=13.0, 8.6 Hz, 2H), 7.46 (dd, J=13.0, 8.6 Hz, 2H), 3.78 (s, 3H), 3.74 (s, 3H) ppm; MS [M+H]⁺ 221, [2M+H]⁺ 441.

### Step 3a-3. Preparation of dimethyl[4-(4,4,5,5-tetramethyl-1,3,2-dioxaborolan-2-yl)phenyl]phosphonate (G2)

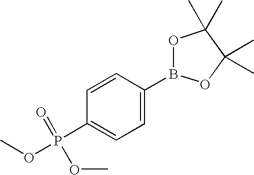

This reaction was performed using a PersonalChemistry™ microwave instrument set at normal absorbance, fixed hold time and 30 sec pre-stirring. A reaction vial was charged with bis(dibenzylidineacetone)palladium(0) (0.13 g, 0.23 mmol) and tricyclohexylphosphine (0.16 g, 0.57 mmol) in dry dioxane (1.0 mL) and the mixture was stirred 30 min under an atmosphere of nitrogen at room temperature. Dimethyl (4-chlorophenyl)phosphonate (0.50 g, 2.26 mmol), bis(pinacolato)diboron (0.60 g, 2.36 mmol) and potassium acetate (0.25 g, 2.54 mmol) were mixed in dry dioxane (5.0 mL) at room temperature under a nitrogen atmosphere in a 10 mL microwave reaction vial and nitrogen was bubbled through the stirred solution for 10 min. The palladium catalyst solution was added and the vial was sealed. The vial was heated at 160° C. for 20 min in the microwave instrument using the conditions listed above. The reaction mixture was filtered through Celite® and the solvent was removed by rotary evaporation under reduced pressure. Chromatography over silica gel using ethyl acetate-hexane (gradient: 0% ethyl acetate to 80%) gave dimethyl[4-(4,4,5,5-tetramethyl-1,3,2-dioxaborolan-2-yl)phenyl]phosphonate (0.34 g, 48% yield) as a colorless oil; ¹H NMR (300 MHz, CDCl₃) δ 7.92-7.88 (m, 2H), 7.82-7.75 (m, 2H), 3.77 (s, 3H), 3.73 (s, 3H), 1.35 (s, 12H) ppm; MS [M+H]⁺ 312, [2M+H]⁺ 625.

### Step 3b-3. Preparation of [4-(4,4,5,5-tetramethyl-1,3,2-dioxaborolan-2-yl)phenyl]phosphonic acid (G3) (Shown in Scheme 3b)

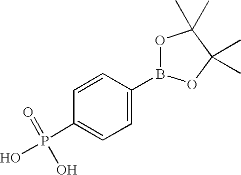

A solution of dimethyl[4-(4,4,5,5-tetramethyl-1,3,2-dioxaborolan-2-yl)phenyl]phosphonate (6.0 g, 19.2 mmol) in dichloromethane (40 mL) was cooled in ice. Bromotrimethylsilane (6.3 mL, 47.8 mmol) was added drop-wise over 2 min and stirred for 2 h at 0° C. Water (1.0 mL, 55.6 mmol) was added and the solution was stirred for 1 h at room temperature. The organic layer was decanted off and the solvent was removed by rotary evaporation under reduced pressure. The crude product was crystallized from 1:3 ethyl acetate-hexane to afford [4-(4,4,5,5-tetramethyl-1,3,2-dioxaborolan-2-yl)phenyl]phosphonic acid (4.93 g, 90% yield) as a white solid; ¹H NMR (300 MHz, CD₃OD) δ 7.75-7.86 (m, 4H), 1.34 (s, 12H) ppm.

### Alternate Synthesis of G3

Pinacol ester G1 (210.0 g, 0.742 mol) was dissolved in chlorobenzene (500 mL, 1.48 M) trimethyl phosphite (270.7 mL, 2.23 mol) was added via funnel and the reaction was heated to 110° C. A solution of 1,1′-azobis-cyclohexane carbonitrile (19.9 g, 0.082 mol) and tri-n-butyltin hydride (235.7 mL, 0.85 mol) in chlorobenzene (250 mL) was added to the flask drop-wise over 4.5 hours. The mixture was stirred for 1.5 hours at 110° C. then cooled to room temperature. Potassium fluoride (172.4 g, 2.97 mol) and water (53.42 ml, 2.97 mol) were added and reaction was for 18 hours at ambient temperature. Sodium sulfate (50 g) was added, and the mixture was filtered through a pad of Celite® and sodium sulfate, washed with dichloromethane (2×750 ml) and concentrate under vacuum to obtain the dimethyl phosphonate, G2, as a yellow solid.

A 3-L flask was charged with G2 (theory 0.742 mol) and anhydrous dichloromethane (740 ml, 1.0 M) and followed by addition of bromotrimethylsilane (225.2 ml, 1.71 mol) via additional funnel. The mixture was stirred at ambient temperature for 2 hours then water (53.2 ml, 3.34 mol) was added and the mixture was stirred for additional hour. The mixture was concentrated to give the crude phosphonic acid, G3, as yellow colored solid. The crude product was recrystallized in 750 ml of tert-butyl methyl ether at 60° C. and cooled to ambient temperature overnight. The suspension was stirred in an ice-water bath for 2 hour and filtered to give pure [4-(4,4,5,5-tetramethyl-1,3,2-dioxaborolan-2-yl)phenyl]phosphonic acid (132.5 g, 63.1% yield). The mother liquor was concentrated then recrystallized in acetonitrile (750 ml) at 60° C. and cooled to ambient temperature and filtered to give 39.7 g of pure [4-(4,4,5,5-tetramethyl-1,3,2-dioxaborolan-2-yl)phenyl]phosphonic acid, G3 (18.9% yield, total yield 172.2 g 82%).

We claim:

1. A process for preparing a compound of structure 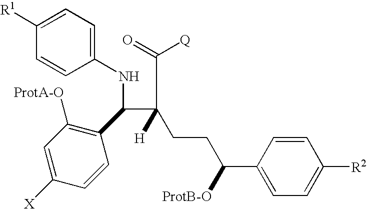
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA-O— is a protecting group for a phenol chosen from an oxymethyl ether, an allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
Q is a chiral auxiliary chosen from single enantiomers of triphenyl glycol and cyclic and branched nitrogen-containing moieties possessing at least one chiral center
said process comprising reacting a compound of formula 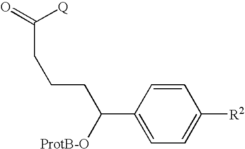
with a compound of formula

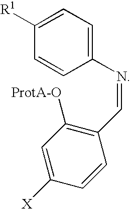

2. A process according to claim 1 for preparing a compound of structure 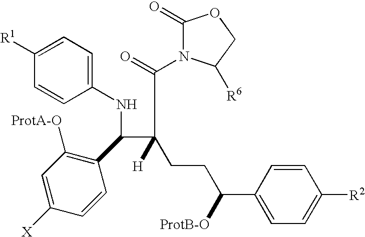
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA-O— is a protecting group for a phenol chosen from an oxymethyl ether, allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
R⁶ is phenyl or benzyl;
said process comprising reacting a compound of formula 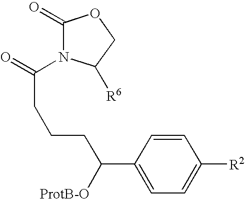
with a compound of formula

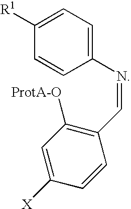

3. A process according to claim 2 comprising reacting a compound of formula 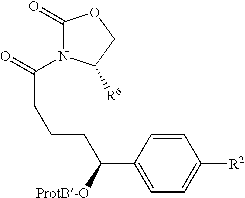
wherein
ProtB′-O— is a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester,
with a Lewis acid and a compound of formula

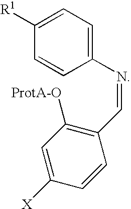

4. A process according to claim 2 comprising the sequential steps of
a. reacting a compound of formula 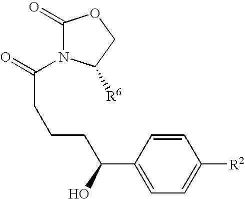
with a trialkylhalosilane in the presence of a base, followed by
b. a Lewis acid, followed by
c. a compound of formula

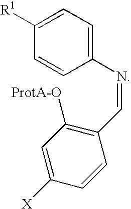

5. A process according to claim 2 wherein
R¹ and R² are chosen from H and halogen; and
ProtA-O— is chosen from methoxymethyl ether, allyl ether, t-butyl ether, benzyl ether, trimethylsilyl ether, t-butyldimethylsilyl ether and t-butyldiphenylsilyl ether.

6. A process according to claim 4 wherein said Lewis acid is a halide of a Group 3, 4, 13 or 14 metal.

7. A process according to claim 6 wherein said Lewis acid is titanium tetrachloride.

8. A process according to claim 4 wherein
R¹ is hydrogen;
R² is fluorine;
X is bromine; and
ProtA-O— is benzyl ether.

9. A process according to claim 2 comprising
a. reacting a compound of formula 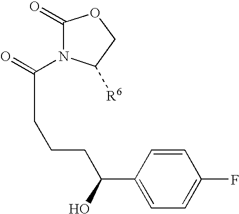
with trimethylchlorosilane in the presence of a tertiary amine to provide a silyl-protected benzyl alcohol; and
b. reacting said silyl-protected benzyl alcohol with titanium tetrachloride and an imine of formula 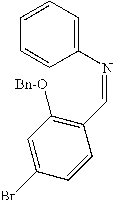
to provide a compound of formula

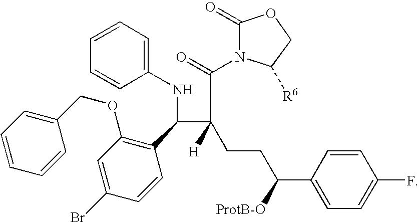

10. A process for preparing a compound of structure 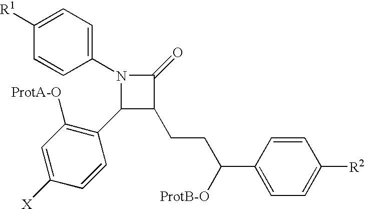
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA-O— is a protecting group for a phenol chosen from an oxymethyl ether, allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester;
said process comprising cyclizing a compound of formula 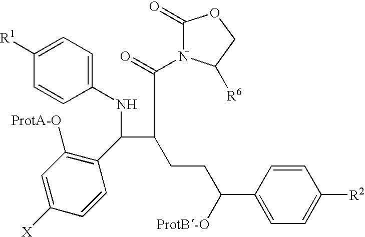
and when ProtB-O is OH, cleaving ProtB′-O—,
wherein
R⁶ is phenyl or benzyl; and
ProtB′-O— is a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester.

11. A process according to claim 10 comprising reacting a compound of formula 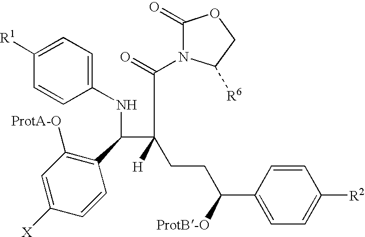
with N,O-bistrimethylsilylacetamide and a source of fluoride ion.

12. A process according to claim 11 wherein said source of fluoride ion is tetrabutylammonium fluoride.

13. A process according to claim 12 wherein
R¹ is hydrogen;
R² is fluorine;
X is bromine;
ProtA is benzyl; and
ProtB′ is silyl.

14. A process according to claim 13 wherein ProtB′ is chosen from t-butyldimethylsilyl and trimethylsilyl.

15. A process for preparing a 4-(biphenylyl)ylazetidinone of formula 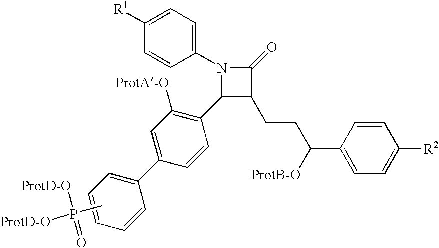
wherein
R¹ and R² are chosen from H, halogen, —OH, and methoxy;
ProtA′-O— is a protecting group for a phenol chosen from an oxymethyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
ProtD-O— is HO— or a protecting group for a phosphonic acid chosen from an alkyl ester, a phenyl ester and a benzyl ester;
said process comprising reacting a 4-phenylazetidin-2-one of formula 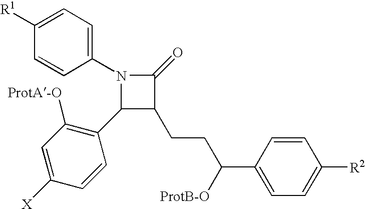
wherein
X is chosen from iodine, bromine, chlorine, toluenesulfonyl, methanesulfonyl and trifluoromethanesulfonyl;
with a phenyl component of formula 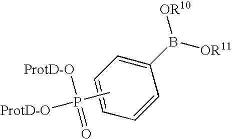
wherein
R¹⁰ and R¹¹ are independently selected from H and (C₁-C₆) alkyl, or R¹⁰ and R¹¹ together form a 5-6 membered ring.

16. A process for preparing a 4-(biphenylyl)azetidinone of formula 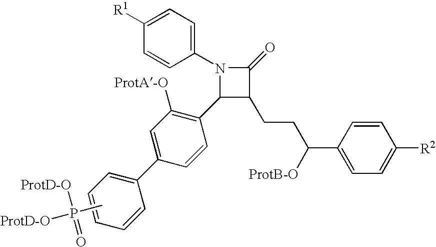
wherein
R¹ and R² are chosen from H, halogen, —OH, and methoxy;
ProtA′-O— is a protecting group for a phenol chosen from an oxymethyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
ProtD-O— is HO— or a protecting group for a phosphonic acid chosen from an alkyl ester, a phenyl ester and a benzyl ester;
said process comprising reacting a 4-phenylazetidin-2-one of formula 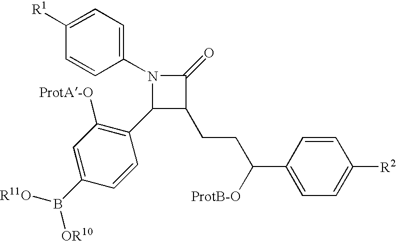
wherein
R¹⁰ and R¹¹ are independently selected from H and (C₁-C₆) alkyl, or R¹⁰ and R¹¹ together form a 5-6 membered ring;
with a phenyl component of formula 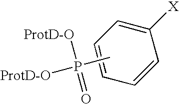
wherein
X is chosen from iodine, bromine, chlorine, toluenesulfonyl, methanesulfonyl and trifluoromethanesulfonyl.

17. A process according to claim 15 wherein said reacting a 4-phenylazetidin-2-one with a phenyl component is carried out with a phosphine, a palladium salt and a base.

18. A process according to claim 15 comprising reacting a 4-phenylazetidin-2-one of formula 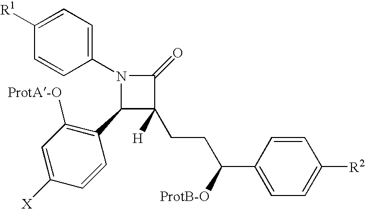
wherein
ProtA′-O— is chosen from methoxymethyl ether, t-butyl ether, silyl ether, and benzyl ether; and
ProtB-O— is chosen from HO— and silyl ether;
with 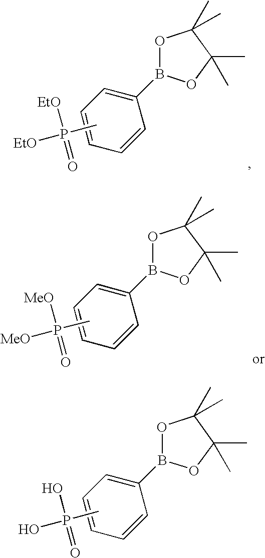
in the presence of a phosphine, a palladium salt and a base.

19. A process according to claim 16 comprising reacting a 4-phenylazetidin-2-one of formula 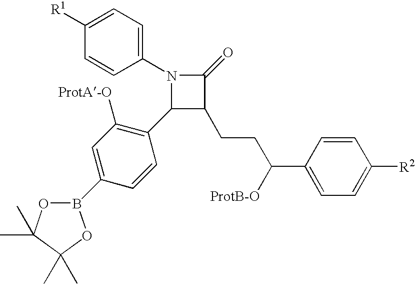
wherein
ProtA′-O— is chosen from methoxymethyl ether, t-butyl ether, silyl ether, and benzyl ether; and
ProtB-O— is chosen from HO— and silyl ether;
with 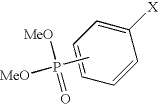
in the presence of a phosphine, a palladium salt and a base.

20. (canceled)

21. A process according to claim 15 wherein R¹ is hydrogen and R² is fluorine.

22. A process for preparing a compound of formula 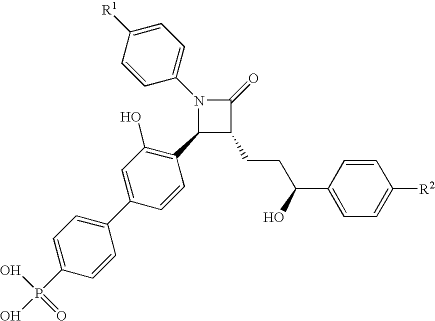
comprising reacting an azetidinone of formula 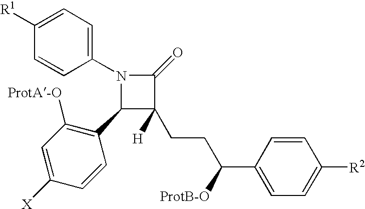
with a dioxaborole of formula 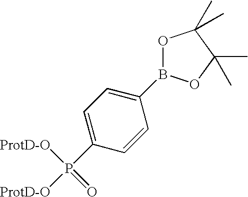
and deprotecting,
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA′-O— is a protecting group for a phenol chosen from an oxymethyl ether, a tertiary alkyl ether, a benzyl ether and a silyl ether;
ProtB-O— is —OH or silyl ether; and
ProtD-O— is —OH, —OCH₃ or —OCH₂CH₃.

23. A process according to claim 22 for preparing 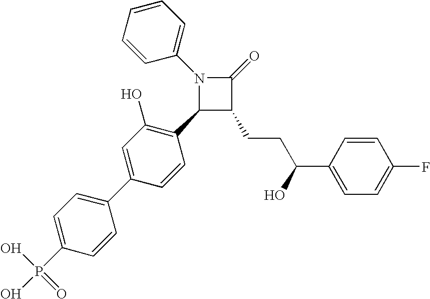
comprising reacting an azetidinone of formula 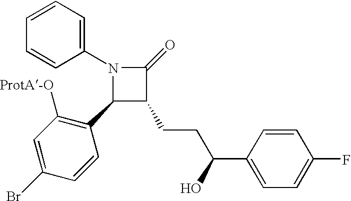
wherein ProtA′ is benzyl or t-butyldimethylsilyl, with a dioxaborole of formula 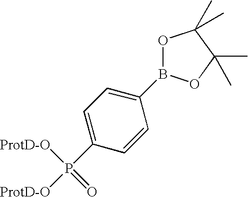
and deprotecting.

24. A process according to claim 22 wherein said azetidinone is reacted with said dioxaborole in the presence of a phosphine, a palladium salt and an alkali metal carbonate;
ProtA′ is benzyl and said deprotection is accomplished by hydrogenolysis with hydrogen gas and palladium on carbon; and
ProtD is H.

25. A process according to claim 22 wherein said azetidinone is obtained by cyclizing a β-aminoacyloxazolinone of formula 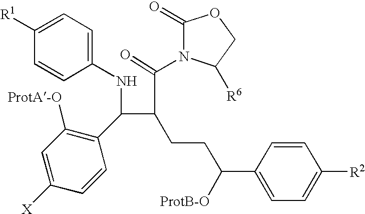
wherein
R⁶ is phenyl or benzyl.

26. A process according to claim 25 wherein said β-aminoacyloxazolinone is obtained by
reacting a compound of formula 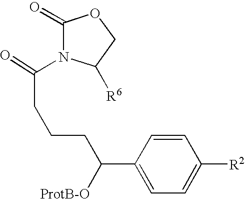
with a compound of formula

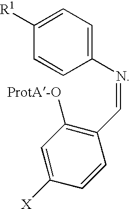

27. A process for preparing an imine of formula 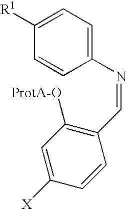
wherein
R¹ is chosen from H, halogen, —OH, and methoxy;
X is chosen from iodine, bromine, chlorine, toluenesulfonyl, methanesulfonyl and trifluoromethanesulfonyl; and
ProtA-O— is a protecting group for a phenol chosen from an oxymethyl ether, allyl ether, a tertiary alkyl ether, a benzyl ether and a silyl ether,
said process comprising reacting a phenol of formula 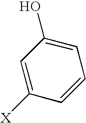
with a source of formaldehyde followed by Schiff base formation by reacting with an aniline of formula 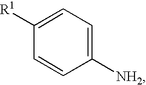
followed by protecting with ProtA.

28. A process according to claim 27 wherein ProtA is benzyl, X is bromine and R¹ is hydrogen.

29. A compound of formula: 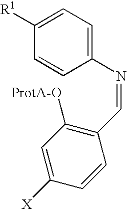
wherein
R¹ is chosen from H, halogen, —OH, and methoxy;
X is chosen from iodine, bromine, chlorine, toluenesulfonyl, methanesulfonyl and trifluoromethanesulfonyl; and
ProtA-O— is a protecting group for a phenol chosen from an oxymethyl ether, allyl ether, a tertiary alkyl ether, a benzyl ether and a silyl ether, with the proviso that when
ProtA- is benzyl, R¹ is H and X is Br, the compound is solid and greater than 95% pure.

30. A compound according to claim 29 wherein R¹ is H or fluoro; X is bromine; and
ProtA-O— is a benzyl ether or silyl ether.

31. A compound of formula 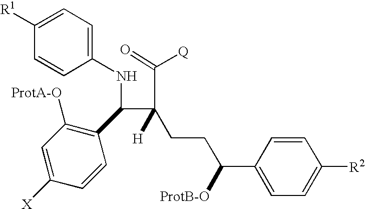
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA-O— is a protecting group for a phenol chosen from an oxymethyl ether, an allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
Q is a chiral auxiliary attached at nitrogen, said chiral auxiliary chosen from single enantiomers of cyclic and branched nitrogen-containing moieties possessing at least one chiral center.

32. A compound according to claim 31 of formula 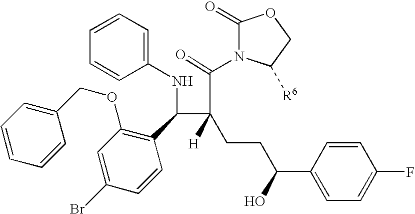
wherein R⁶ is phenyl or benzyl.

33. A compound of formula 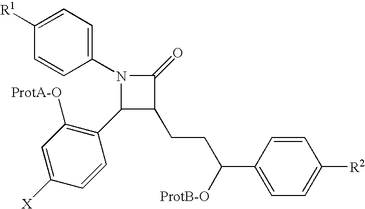
wherein
R¹ and R² are chosen from H, halogen, —OH, and methoxy;
X is chosen from iodine, bromine, chlorine, toluenesulfonyl, methanesulfonyl and trifluoromethanesulfonyl;
ProtA-O— is a protecting group for a phenol chosen from an oxymethyl ether, allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester.

34. A compound according to claim 33 of formula

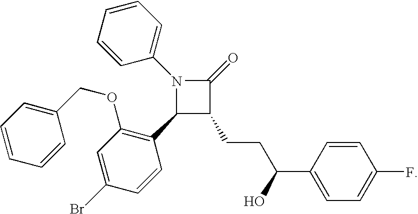

35. A compound of formula 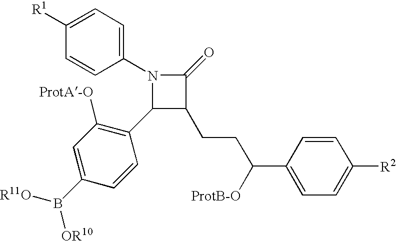
wherein
R¹ and R² are chosen from H, halogen, —OH, and methoxy;
ProtA′-O— is a protecting group for a phenol chosen from an oxymethyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
R¹⁰ and R¹¹ are independently selected from H and (C₁-C₆) alkyl, or R¹⁰ and R¹¹ together form a 5-6 membered ring;

36. A compound according to claim 35 of formula

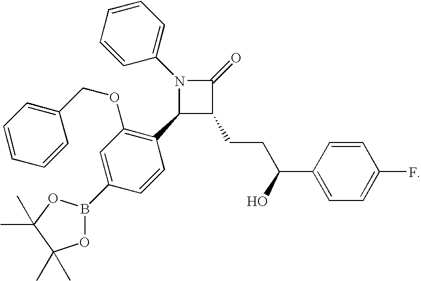

37. A compound of formula 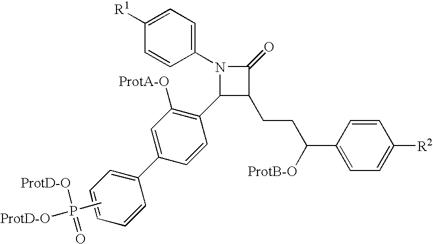
wherein
R¹ and R² are chosen from H, halogen, —OH, and methoxy;
ProtA-O— is a protecting group for a phenol chosen from an oxymethyl ether, allyl ether, a tertiary alkyl ether, a benzyl ether and a silyl ether;
ProtB-O— is HO— or a protecting group for a benzylic alcohol chosen from an oxymethyl ether, a tetrahydropyranyl or tetrahydrofuranyl ether, methoxycyclohexyl ether, a methoxybenzyl ether, a silyl ether and an ester; and
ProtD-O— is HO— or a protecting group for a phosphonic acid chosen from an alkyl ester, a phenyl ester and a benzyl ester.

38. A compound according to claim 37 chosen from 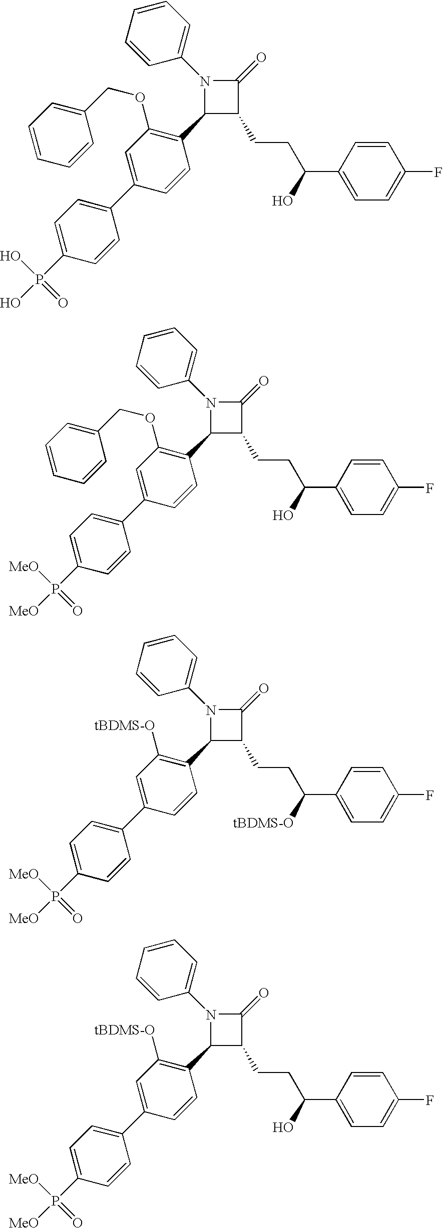 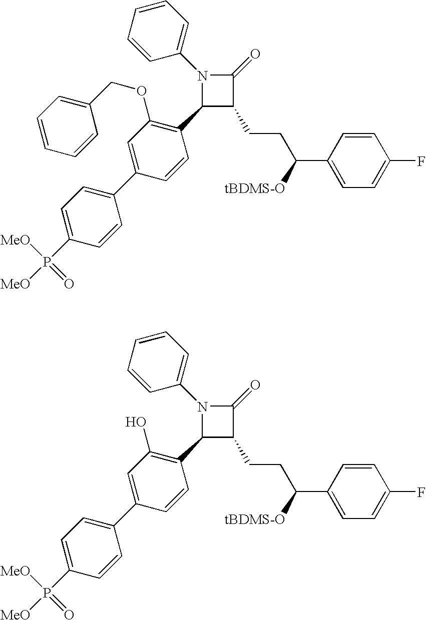
and salts and solvates thereof.

39. (3′-(Benzyloxy)-4′-{(2S,3R)-3-[(3S)-3-(4-fluorophenyl)-3-hydroxypropyl]-4-oxo-1-phenylazetidin-2-yl}biphenyl-4-yl)phosphonic acid dicyclohexylammonium salt according to claim 38.

40. A process for preparing [4-(4,4,5,5-tetramethyl-1,3,2-dioxaborolan-2-yl)phenyl]phosphonic acid
comprising 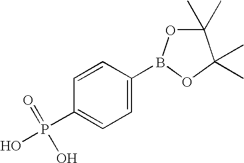
(1) reacting 4-bromo-(4,4,5,5-tetramethyl-1,3,2-dioxaborolan-2-yl)benzene with trimethylphosphine in the presence of a catalytic amount of a radical initiator followed by reduction to provide dimethyl[4-(4,4,5,5-tetramethyl-1,3,2-dioxaborolan-2-yl)phenyl]phosphonate;
(2) hydrolyzing dimethyl[4-(4,4,5,5-tetramethyl-1,3,2-dioxaborolan-2-yl)phenyl]phosphonate by treatment with bromotrimethylsilane followed by water; and
(3) isolating [4-(4,4,5,5-tetramethyl-1,3,2-dioxaborolan-2-yl)phenyl]phosphonic acid.

41. A process according to claim 40 wherein said radical initiator is 1,1′-azobis(cyclohexanecarbonitrile) and said reduction is accomplished by treatment with tributyltin hydride or tris(trimethylsilyl) silane.

42. The compound of claim 34, as a crystalline solid of greater than 99% diastereomeric purity, melting above 113° C.

43. The compound of claim 34 characterized in that the compound is obtained by a process of crystallization from isopropyl alcohol/water.

44. A process according to claim 16 wherein R¹ is hydrogen and R² is fluorine.
